(19) United States
(12) Patent Application Publication
(10) Pub. No.: US 2022/0144926 A1
(43) Pub. Date: May 12, 2022

(54) IDENTIFICATION AND TARGETING OF PATHOGENIC EXTRACELLULAR MATRIX FOR DIAGNOSIS AND TREATMENT OF CANCER AND OTHER DISEASES

(71) Applicant: Board of Regents, The University of Texas System, Austin, TX (US)

(72) Inventors: Raghu KALLURI, Houston, TX (US); Valerie LEBLEU, Houston, TX (US); Yang CHEN, Houston, TX (US)

(21) Appl. No.: 17/286,206

(22) PCT Filed: Oct. 16, 2019
(86) PCT No.: PCT/US2019/056576

§ 371 Date: Apr. 16, 2021

Related U.S. Application Data
(60) Provisional application No. 62/746,286, filed on Oct. 16, 2018.

(51) Int. Cl. C07K 16/18; C07K 16/28; A61K 45/06; A61K 35/17; A61P 35/00; G01N 33/574

(52) U.S. Cl. CPC C07K 16/18; C07K 16/2809; A61K 45/06; A61K 35/17; G01N 2333/78; G01N 33/57438; C07K 2317/33; C07K 2317/31; C07K 2319/03; A61P 35/00

(57) ABSTRACT

Provided herein are agents, such as antibodies or chimeric antigen receptors, that target homotrimeric type I collagen. Methods of treating cancer and fibroids are provided, comprising administering to a patient in need thereof an effective amount of a homotrimeric type I collagen-neutralizing agent. The methods can further include administering an effective amount of chemotherapy or immunotherapy to said patient.

### REFERENCE TO RELATED APPLICATIONS

The present application claims the priority benefit of U.S. provisional application No. 62/746,286, filed Oct. 16, 2018, the entire contents of which is incorporated herein by reference.

### BACKGROUND

### 1. Field

The present invention relates generally to the field of medicine. More particularly, it concerns methods of detecting cancer based on the presence of homotrimeric type I collagen and treating cancer by disrupting homotrimeric type I collagen and signaling induced thereby.

### 2. Description of Related Art

Desmoplasia, dense stroma composed of various cell populations such as myofibroblasts, and deposition of extracellular matrix (ECM), such as type I collagen (Col1), are the defining features of pancreatic ductal carcinoma (PDAC). However, the specific roles of ECM and tumor stroma in supporting or restraining tumorigenesis are still controversial (Mueller and Fusenig, 2004; Neesse et al., 2015). There have been observations supporting both tumor-supporting and tumor-restraining contributions of PDAC stroma. Previous observations demonstrated that the desmoplastic stroma (such as activated pancreatic stellate cells [PSCs]/myofibroblasts and ECM) in PDAC forms the pro-tumorigenic microenvironment, which contributes to compromised drug delivery and therapy resistance. Targeting PDAC stroma has been demonstrated to reduce PDAC desmoplasia and improve drug delivery by inhibition of the sonic hedgehog (SHH) pathway (Olive et al., 2009) or by ablating stromal hyaluronic acid (Provenzano et al., 2012). However, clinical trials targeting PDAC stroma failed to yield promising therapeutic outcome as expected. Additionally, recent studies argue for the heterogeneity of stromal fibroblasts and their multiple roles (Ohlund et al., 2014; Kalluri, 2016; Ohlund et al., 2017). Previous studies have shown that depletion of proliferating α-smooth muscle actin (αSMA)-expressing activated PSCs/myofibroblasts elicits the hypoxic status and invasiveness of PDAC, despite the decreased fibrosis and collagen deposition in PDAC stromal (Ozdemir et al., 2014). Genetic ablation of SHH or smoothened inhibition also lead to more aggressive and less differentiated PDAC. These arguments are concordant with earlier studies suggesting the restraining function of tumor stroma (Rhim et al., 2014). It has also been reported that the response of PDAC toward anti-stromal therapies may vary greatly due to the different genotypes and signaling of PDAC, which largely determine stromal remodeling (Laklai et al., 2016). Collectively, these various, or even conflicting, observations indicate a complex biology and multiplex roles of PDAC stroma beyond previous knowledge, which undoubtedly requires further systematic investigations using new experimental systems.

Current genetically engineered mouse models (GEMMs) of PDAC, such as the classic KPC (LSL-Kras^{G12D/+}; Trp53^{R172H/+} or Trp53^{loxP/loxP}; Pdx1-Cre) model, have provided valuable platforms mimicking the clinical situation of human PDAC, and have enormously contributed to the research on PDAC and its therapeutics (Hingorani et al., 2005). The conventional KPC models have been extensively used in combination with genetic ablation of floxed (flanked by loxP sites) genes in cancer cells (using the same pancreatic-specific Cre, such as Pdx1-Cre or P48-Cre), or with whole-body knockout (KO) of genes. However, it remains impossible to achieve cell-type-specific genetic manipulations in stromal cell subpopulations (such as myofibroblasts or immune cells) in these GEMMs, due to the universal Cre-loxP recombination mechanism. And it is also impossible to establish KPC models containing the whole-body KO of those genes with KO lethality, for instance, Col1a1 that encodes type I collagen α1 chain (Lohler et al., 1984). Thus, it is surprising, yet reasonable, that so far there is no PDAC GEMM that enables the functional KO of Col1 in stromal cell source(s) to testify to the origin and contribution of Col1, especially considering Col1 is such an essential component and the most abundant protein in the PDAC desmoplasia and microenvironment.

Type I collagen (Col1), normally composed of α1 chain and a2 chain, is one of the most dominantly deposited interstitial ECM components in PDAC microenvironment. Many studies have indicated that activated PSCs/myofibroblasts are the major cell source of Col1 as well other ECM materials (Haber et al., 1999; Armstrong et al., 2004; Bachem et al., 2005; Fujita et al., 2009; Apte et al., 2012). Nevertheless, Col1 has also been shown to be produced by various types of cancer cells and to promote tumor progression. In fact, cancer cell-derived Col1 consists of unique and MMP-resistant homotrimer (α1)₃ chains, in contrary to the (α1/α2/α1) heterotrimer chains produced by fibroblasts or other normal cells (Sengupta et al., 2003; Han et al., 2008; Egeblad et al., 2010; Han et al., 2010; Makareeva et al., 2010). These observations indicated the distinct structures and functional roles of cancer-derived Col1 versus myofibroblast-derived Col1 in cancer. Numerous studies have previously been conducted to address the active roles of PDAC stroma. However, the roles of ECM components, such as Col1, with respect to the specific cell origins have not been systematically verified or compared in clinically relevant transgenic PDAC models. To further understand the influence of stroma on PDAC development, it is important to dissect the precise functions of Coil specifically derived from various cellular origins, such as cancer cells and fibroblast subpopulations.

### SUMMARY

In one embodiment, provided herein are antibodies or antibody fragments that bind to α1 homotrimeric type I collagen. In some aspects, the antibodies or antibody fragments have an affinity for α1 homotrimeric type I collagen that is at least two, three, four, five, six, seven, eight, nine, or ten times higher than an affinity for α1/α2/α1 heterotrimeric type I collagen. In some aspects, the antibodies or antibody fragments do not detectably bind to α1/α2/α1 heterotrimeric type I collagen. The antibodies or antibody fragments may recognize a conformations or specific discontinuous epitope that is present in the homotrimer but not the heterotrimer.

In some aspects, the antibody fragments are recombinant scFv (single chain fragment variable) antibodies, Fab fragments, F(ab′)₂ fragments, or Fv fragments. In some aspects, the antibodies are chimeric antibodies or bispecific antibodies. In certain aspects, the chimeric antibodies are humanized antibodies. In certain aspects, the bispecific antibodies bind to both α1 homotrimeric type I collagen and CD3. In some aspects, the antibodies or antibody fragments are conjugated to a cytotoxic agent. In some aspects, the antibodies or antibody fragments are conjugated to a diagnostic agent.

In one embodiment, provided herein are hybridomas or engineered cells encoding antibodies or antibody fragments of the present embodiments. In some embodiments, pharmaceutical formulations are provided that comprise one or more of the antibodies or antibody fragments of the present embodiments.

In one embodiment, provided herein are methods of treating a patient in need thereof, the method comprising administering an effective amount of an α1 homotrimeric type I collagen-specific antibody or antibody fragment. In some aspects, the α1 homotrimeric type I collagen-specific antibody or antibody fragment is the antibody or antibody fragment of any of the present embodiments.

In some aspects, the patient has a cancer, a fibroid disease, keloids, organ fibrosis, Crohn's disease, strictures, colitis, psoriasis, or a connective tissue disorder. In some aspects, the connective tissue disorder is a connective tissue disorder that involves collagen. In certain aspects, the connective tissue disorder that involves collagen is a connective tissue disorder that involved type 1 collagen.

In some aspects, the patient has a cancer. In some aspects, the cancer patient has been determined to express an elevated level of α1 homotrimeric type I collagen relative to a control patient. In certain aspects, the cancer is a pancreatic cancer. In some aspects, the methods are further defined as methods of inhibiting pancreatic cancer metastasis. In some aspects, the methods are further defined as methods of inhibiting pancreatic cancer growth. In some aspects, the methods further comprise administering at least a second anti-cancer therapy. In certain aspects, the second anti-cancer therapy is a chemotherapy, immunotherapy, radiotherapy, gene therapy, surgery, hormonal therapy, anti-angiogenic therapy or cytokine therapy.

In one embodiment, provided herein are chimeric antigen receptor (CAR) polypeptides comprising, from N- to C-terminus, an antigen binding domain; a hinge domain; a transmembrane domain and an intracellular signaling domain, wherein the CAR polypeptide binds to an α1 homotrimeric type I collagen. In some aspects, the antigen binding domain comprises HCDR sequences from a first antibody that binds to an α1 homotrimeric type I collagen and LCDR sequences from a second antibody that binds to an α1 homotrimeric type I collagen. In some aspects, the antigen binding domain comprises HCDR sequences and LCDR sequence from an antibody that binds to an α1 homotrimeric type I collagen. In some aspects, the antigen binding domain has an affinity for α1 homotrimeric type I collagen that is at least two, three, four, five, six, seven, eight, nine, or ten time higher than an affinity for α1/α2/α1 heterotrimeric type I collagen. In some aspects, the antigen binding domain does not detectably bind to α1/α2/α1 heterotrimeric type I collagen.

In some aspects, the hinge domain is a CD8a hinge domain or an IgG4 hinge domain. In some aspects, the transmembrane domain is a CD8a transmembrane domain or a CD28 transmembrane domain. In some aspects, the intracellular signaling domain comprises a CD3z intracellular signaling domain.

In one embodiment, provided herein are nucleic acid molecules encoding a CAR polypeptide of any of the present embodiments. In some aspects, the sequence encoding the CAR polypeptide is operatively linked to expression control sequences.

In one embodiment, provided herein are isolated immune effector cells comprising a CAR polypeptide or nucleic acid of the present embodiments. In some aspects, the nucleic acid is integrated into the genome of the cell. In some aspects, the cell is a T cell. In some aspects, the cell is an NK cell. In some aspects, the cell is a human cell. In one embodiment, provided herein are pharmaceutical compositions comprising a population of cells of the present embodiments in a pharmaceutically acceptable carrier.

In one embodiment, provided herein are methods of treating a subject comprising administering an anti-tumor effective amount of chimeric antigen receptor (CAR) T cells that expresses a CAR polypeptide in accordance with any one of the present embodiments. In some aspects, the CAR T cells are allogeneic cells. In some aspects, the CAR T cells are autologous cells. In some aspects, the CAR T cells are HLA matched to the subject. In some aspects, the subject has a cancer, such as, for example, a pancreatic cancer. In some aspects, the methods further comprise administering a demethylating drug prior to administering the CAR T cells, in order to serve as a primer for immunotherapy. The demethylating drug may reverse Col1A2 hypermethylation. The demethylating drug may be 5-azacytidine or 5-aza-2′-deoxycytidine. The In some aspects, the methods further comprise administering a drug that interferes with the methylation of promoters of the Col1A2 gene.

In one embodiment, provided herein are methods of treating a subject comprising administering an anti-tumor effective amount of chimeric antigen receptor (CAR) NK cells that expresses a CAR polypeptide in accordance with any one of the present embodiments. In some aspects, the CAR NK cells are allogeneic cells. In some aspects, the CAR NK cells are autologous cells. In some aspects, the CAR NK cells are HLA matched to the subject. In some aspects, the subject has a cancer, such as, for example, a pancreatic cancer. In some aspects, the methods further comprise administering a demethylating drug prior to administering the CAR NK cells, in order to serve as a primer for immunotherapy. The demethylating drug may reverse Col1A2 hypermethylation. The demethylating drug may be 5-azacytidine or 5-aza-2′-deoxycytidine. The In some aspects, the methods further comprise administering a drug that interferes with the methylation of promoters of the Col1A2 gene.

In one embodiment, provided herein are methods of diagnosing a patient as having a disease, the method comprising contacting a cancer tissue obtained from the subject with an antibody of any one of the present embodiments and detecting the binding of the antibody to the tissue, wherein if the antibody binds to the tissue, then the patient is diagnosed as having a cancer or a fibroid disease. In some aspects, the disease is a cancer, a fibroid disease, keloids, organ fibrosis, Crohn's disease, strictures, colitis, psoriasis, or a connective tissue disorder. In some aspects, the connective tissue disorder is a connective tissue disorder that involves collagen. In some aspects, the connective tissue disorder that involves collagen is a connective tissue disorder that involved type 1 collagen.

In one embodiment, provided herein are methods of classifying a patient having pancreatic ductal adenocarcinoma, the method comprising determining a type I collagen/CK19 ratio in a cancer tissue obtained from the subject, wherein a ratio that is lower than a ratio in a reference normal tissue indicates that the patient has a more advanced disease status. In some aspects, the reference normal tissue is obtained from the patient.

In one embodiment, provided herein are methods of treating a subject having a disease, the method comprising administering an anti-tumor effective amount of a composition that inhibits an enzyme that crosslinks α1 type I collagen homotrimers. In one embodiment, provided herein are methods of treating a subject having a disease, the method comprising administering an anti-tumor effective amount of a composition that inhibits a chaperone that promotes that formation of α1 type I collagen homotrimers. In one embodiment, provided herein are methods of treating a subject having a disease, the method comprising administering an anti-tumor effective amount of a composition that inhibits pro-oncogenic signaling through the DDR1 receptor. In some aspects, the subject has been determined to express an elevated level of α1 homotrimeric type I collagen relative to a control subject.

In some aspects, the disease is a cancer, a fibroid disease, keloids, organ fibrosis, Crohn's disease, strictures, colitis, psoriasis, or a connective tissue disorder. In some aspects, the connective tissue disorder is a connective tissue disorder that involves collagen. In some aspects, the connective tissue disorder that involves collagen is a connective tissue disorder that involved type 1 collagen.

In some aspects, the disease is a cancer. In certain aspects, the cancer is a pancreatic cancer. In some aspects, the methods are further defined as methods of inhibiting pancreatic cancer metastasis. In some aspects, the methods are further defined as methods of inhibiting pancreatic cancer growth. In some aspects, the methods further comprise administering at least a second anti-cancer therapy. In certain aspects, the second anti-cancer therapy is a chemotherapy, immunotherapy, radiotherapy, gene therapy, surgery, hormonal therapy, anti-angiogenic therapy or cytokine therapy.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. FIG. 1A. Genetic strategy to delete type I collagen α1 (Col1α1) specifically in αSMA-expressing cell population in the context of pancreatic cancer using the KPPF;αSMA-Cre;Col1a1^{loxP/loxP} (referred to as KPPF;Col1^{smaKO}) mice. Littermates with KPPF (KPPF;Cre-negative;Col1a1^{loxP/loxP}) genotype were used as control mice. FIG. 1B. Serial sections of pancreatic tumors from KPPF or KPPF;Col1^{smaKO} mice, stained with hematoxylin and eosin (H&E), Picrosirius Red, MTS, Col1 (immunohistochemistry), and αSMA (immunohistochemistry).

FIGS. 2A-F. FIG. 2A. Representative atomic force microscopic (AFM) images of cryosections of KPPF and KPPF;Col1^{smaKO} tumors. Quantification of the elastic modulus was based on the AFM assays of 3 mice per group. FIG. 2B. Survival curves of KPPF and KPPF;Col1^{smaKO} mice. FIG. 2C. Percentage of mice showing abdominal swelling and ascites in indicated groups. FIG. 2D. Serial sections of pancreas of KPPF or KPPF;Col1^{smaKO} mice at PanIN or PDAC stage, stained by H&E, CK19 (immunohistochemistry), and Col1 (immunohistochemistry). Quantification of the Col1/CK19 ratio was calculated based on the percent positive area of CK19 and Col1. FIG. 2E. GSEA-Hallmark enrichment analysis, shown with normalized enrichment score (NES), for Most significantly up-regulated cell signaling pathways based on the GSEA-Hallmark enrichment analysis of the whole transciptome RNA Sequencing (RNA-Seq) data from KPPF tumors (n=3 mice per group) or KPPF;Col1^{smaKO} tumors (n=4 mice per group). FIG. 2F. Q-PCR analysis for Col1α1 in MFs.

FIGS. 3A-F. FIG. 3A. Genetic strategy to induce oncogenic Kras^{G12D} using the Pdx1-Flp-FRT recombination system in KF (FSF-Kras^{G12D/+};Pdx1-Flp) mice. Type I collagen α1 (Col1a1) was specifically deleted in αSMA-expressing cell population using KF;αSMA-Cre;Col1a1^{loxP/loxP} (referred to as KF;Col1^{smaKO}) mice (see FIG. 10A), or deleted in Pdx1-expressing cancer cell lineage using KF;Pdx1-Cre;Col1a1^{loxP/loxP} (referred to as KF;Col1^{pdxKO}) mice. FIG. 3B. Serial sections of pancreas of KF or KF;Col1^{pdxKO} (age-matched 6-month-old) mice, stained by H&E and Col1 (immunohistochemistry). FIG. 3C. The percentage of ADM and PanIN lesions in pancreas from age-matched 6-month-old mice with KF (left column), KF;Col1^{smaKO} (right column), or KF;Col1^{pdxKO} (middle column) genotypes. FIG. 3D. Col1 immunohistochemistry staining in ADM lesions of KF (left column) and KF;Col1^{pdxKO} (right column) mice. FIGS. 3E&F. Sox9 positivity (%) in ADM and PanIN lesions of KF (left column) and KF;Col1^{pdxKO} (right column) mice (FIG. 3E). Representative images of Sox9 immunohistochemistry staining were shown in (FIG. 3F).

FIGS. 4A-F. FIG. 4A. Genetic strategy to delete type I collagen α1 (Col1a1) in cancer cell lineage in the context of pancreatic cancer using the LSL-Kras^{G12D};Trp53^{loxP/loxP}; Pdx1-Cre;Col1a1^{loxP/loxP} (referred to as KPPC;Col1^{pdxKO}) mice. The LSL-Kras^{G12D};Trp53^{loxP/loxP}; Pdx1-Cre (KPPC) mice were used as control animals. FIG. 4B. Survival of KPPC (the bottom line at the Day 53 time point) and KPPC;Col1^{pdxKO} (the top line at the Day 53 time point) mice. FIG. 4C. Percentage of PanIN lesion areas of KPPC (left column) and KPPC;Col1^{pdxKO} (right column) mice at the same age of 28 days. FIG. 4D. Serial sections of pancreatic tumor sections from KPPC (left column) and KPPC;Col1^{pdxKO} (right column) mice at the same age of 53 days, stained with hematoxylin and eosin (H&E), Col1 (immunohistochemistry), and Picrosirius Red. FIG. 4E. Histology evaluation of tumors from KPPC and KPPC;Col1^{pdxKO} mice at the same age of 53 days. FIG. 4F. Pancreatic tumor burden (tumor weight/body weight) of KPPC (left column) and KPPC;Col1^{pdxKO} (right column) mice at the same age of 53 days.

FIGS. 5A-I. FIGS. 5A-D. Whole transcriptome RNA sequencing (RNA-Seq) analysis was conducted on KPPC tumors (n=4) and KPPC;Col1^{pdxKO} tumors (n=5). GSEA plots were shown with normalized enrichment score (NES) of up-regulated gene clusters based on GSEA-Hallmark enrichment analysis for KPPC;Col1^{pdxKO} tumors (FIG. 5A) and KPPC tumors (FIG. 5B), as summarized in (FIG. 5C). Top up-regulated genes were listed in (FIG. 5D). FIG. 5E. Top up-regulated gene networks identified based on the enriched transcripts in KPPC;Col1^{pdxKO} tumors and KPPC tumors. FIGS. 5F-I. Whole transcriptome RNA sequencing (RNA-Seq) analysis was conducted on KPPC and KPPC;Col1^{pdxKO} cell lines. GSEA plots were shown with normalized enrichment score (NES) of up-regulated gene clusters based on GSEA-Hallmark analysis for KPPC;Col1^{pdxKO} cells (FIG. 5F) and KPPC cells (FIG. 5G), as summarized in (FIG. 5H). Top up-regulated genes were listed in (FIG. 5I).

FIGS. 6A-H. FIG. 6A. Primary mouse cancer cell lines established from KPPC and KPPC;Col1^{pdxKO} tumors. FIG. 6B. Cell proliferation of KPPC (top line) and KPPC;Col1^{pdxKO} (bottom line) cell lines over time. Cell viability of KPPC and KPPC;Col1^{pdxKO} cell lines in the presence of gemcitabine at various concentrations. FIGS. 6C&D. 3D tumor spheroids established from KPPC and KPPC;Col1^{pdxKO} cell lines. Average diameter of spheroids by KPPC (left column) and KPPC;Col1^{pdxKO} (right column) cell lines was quantified in (FIG. 6D). FIG. 6E. Gene expression profile of various collagen types in KPPC (left column of each pair) and KPPC;Col1^{pdxKO} (right column of each pair) cell lines, as examined by qRT-PCR. FIG. 6F. Methylated DNA immunoprecipitation (MeDIP) assay of Col1a1 and Col1a2 genes in primary mouse cancer cell lines established from pancreatic tumors of transgenic mouse models including KF, KPF, KPPF, KPPC, KTC, and PKT strains, as compared to 3T3 mouse fibroblasts. Relative expression levels of Col1a1 and Col1a2 in KPPC primary mouse cancer cell line, as compared to primary mouse fibroblasts sorted from KPPC tumor. Characterization of Col1α1 chain and Col1α2 chain of purified Col1 homotrimers and heterotrimers from cell culture medium of KPPC cancer cells, KPPC;Col1^{pdxKO} cells, and 3T3 fibroblasts, respectively. The sensitivity of Col1 homotrimers and heterotrimers to MMP degradation was examined. FIG. 6G. Whole genome DNA methylation analysis of human pancreatic cancer cell lines and normal human pancreatic epithelial cell line (HPNE). DNA methylation at COL1A1 and COL1A2 gene promoter regions was shown. FIG. 6H. qRT-PCR examination of COL1A1 (left column of each pair) and COL1A2 (right column of each pair) genes in human pancreatic cancer cell lines, as compared with BJ fibroblasts.

FIGS. 7A-J. FIG. 7A. Genetic strategy to induce oncogenic Kras^{G12D} and homozygous p53 loss using the Pdx1-Flp-FRT recombination system in KPPF (FSF-Kras^{G12D/+};Trp53^{frt/frt};Pdx1-F1p) mice. FIG. 7B. Representative pancreatic sections of normal, PanIN, and PDAC stages of KPPF mice, stained by hematoxylin and eosin (H&E) and type I collagen (Col1) immunohistochemistry. FIG. 7C. Genetic strategy to induce oncogenic Kras^{G12D} and homozygous p53 loss using the Pdx1-Cre-loxP recombination system in KPPC (LSL-Kras^{G12D};Trp53^{loxP/loxP}; Pdx1-Cre) mice. FIG. 7D. Representative pancreatic sections of normal, PanIN, and PDAC stages of KPPC mice, stained by hematoxylin and eosin (H&E) and type I collagen (Col1) immunohistochemistry. FIGS. 7E-F. Genetic strategy to induce EGFP expression in Pdx1-Flp lineage and tdTomato expression in αSMA-Cre lineage using a Rosa26-CAG-loxP-frt-Stop-frt-FirefyLuc-EGFP-loxP-RenillaLuc-tdTomato (R26^{Dual}) tracer in KPPF;αSMA-Cre;R26^{Dual} mice. FIG. 7G. Representative images of primary PDAC tumors from KPPF;αSMA-Cre;R26^{Dual} mice examined for intrinsic EGFP (in cancer cells) and tdTomato (in αSMA-expressing myofibroblasts) signals. FIG. 7H. Electrophoretic migration of PCR products of the DNA from primary cell culture of cancer cells and myofibroblasts sorted from KPPF or KPPF;Col1^{smaKO} mice. PCR product detection confirmed the specific deletion of Col1a1 by gene recombination shown by the expected lanes specifically in myofibroblasts from KPPF;Col1^{smaKO} mice. FIG. 7I. Systemic loss of Col1a1 using CMV-Cre, results in embryonic lethality. FIG. 7J. H&E of pancreas and quantification of ADM and PanIN in the indicated groups (KF;Col1^{pdxKO} is the left column; KF;Cre^{reg};Col1^{F/F} is the middle column; KF;Col1^{smaKO} is the right column).

FIGS. 8A-C. FIGS. 8A&B. Serial sections of pancreas of KPPF mice during the disease progression from ADM/early PanIN to PanIN (FIG. 8A), or from PanIN to PDAC (FIG. 8B), stained by H&E, CK19, Col1, and αSMA immunohistochemistry. FIG. 8C. Quantification of the percent positive area of CK19, Col1, and αSMA, or the Col1/CK19 ratio, at each stage of disease progression.

FIG. 9. Overall survival (OS) and progression-free survival (PFS) of pancreatic adenocarcinoma patients from TCGA dataset correlated with the ratio of COL1A1 expression level and CK19 expression level (RNA Seq V2 RSEM). Patients were stratified into two groups based on the median COL1A1/CK19 ratio (or in control panels, by COL1A1/GAPDH ratio or COL1A1/ACTB ratio).

FIGS. 10A-B. FIG. 10A. Genetic strategy to delete type I collagen α1 (Col1a1) specifically in αSMA-expressing cell population in the context of pancreatic cancer using the KF;αSMA-Cre;Col1a1^{loxP/loxP} (referred to as KF;Col1^{smaKO}) mice. Littermates with KF (KF;Cre-negative;Col1a1^{loxP/loxP}) genotype were used as control mice. FIG. 10B. Serial sections of pancreas of KF or KF;Col1^{smaKO} (age-matched 6-month-old) mice, stained by H&E, MTS, Col1 (immunohistochemistry), and αSMA (immunohistochemistry).

FIGS. 11A-B. FIG. 11A. Genetic strategy to delete type I collagen α1 (Col1a1) in cancer cell lineage in the context of pancreatic cancer using the LSL-Kras^{G12D};Pdx1-Cre;Col1a1^{loxP/loxP} (referred to as KC;Col1^{pdxKO}) mice. LSL-Kras^{G12D};Pdx1-Cre (KC) mice were used as control animals. FIG. 11B. Serial sections of pancreatic tumor sections from KC or KC;Col1^{pdxKO} mice, stained with hematoxylin and eosin (H&E), Picrosirius Red, MTS, Col1 (immunohistochemistry), or αSMA (immunohistochemistry).

FIGS. 12A-D. FIG. 12A. Survival of KPPC (bottom line at the Day 60 time point), KPPC;Col1^{pdxKO/+} (heterozygous Col1a1 deletion) (middle line at the Day 60 time point), and KPPC;Col1^{pdxKO} (top line at 60 Day time point) mice. FIG. 12B. Serial sections of pancreatic tumor sections from KPPC and KPPC;Col1^{pdxKO} mice at endpoint stage, stained with hematoxylin and eosin (H&E), Col1 (immunohistochemistry), and Picrosirius Red. FIG. 12C. MeDIP assay of COL1A1 and COL1A2 genes in various human pancreatic cancer cell lines, as compared with BJ fibroblasts. FIG. 12D. Cell viability assay of KPPC and KPPC;Col1^{pdxKO} cells treated with Col1 solution (heterotrimers from rat tails) at indicated concentrations for 48 h.

FIG. 13. (Right panel) Relative expression levels of Col1a1 and Col1a2 in KPPC cancer cells, KPPC;Col1^{pdxKO} cancer cells, and 3T3 mouse fibroblasts treated with demethylation agent 5-Azacytidine (5-AZA). (Left panel) Characterization of Col1α1 chain and Col1α2 chain of purified Col1 homotrimers (from Panci human PDAC cell line) and heterotrimers (from BJ fibroblast line) by Western Blot.

FIGS. 14A-D. FIG. 14A. Genetic strategy to delete type I collagen α1 (Col1a1) specifically in Fsp1-expressing cell population in the context of pancreatic cancer using the KPPF;Fsp1-Cre;Col1a1^{loxP/loxP} (referred to as KPPF;Col1^{fspKO}) mice. Littermates with KPPF (KPPF;Cre-negative;Col1a1^{loxP/loxP}) genotype were used as control mice. FIG. 14B. Survival of KPPF and KPPF;Col1^{fspKO} mice. FIG. 14C. Relative expression level of Col1a1 in Fsp1-antibody-sorted fibroblasts from KPPF and KPPF;Col1^{fspKO} tumors. These fibroblasts were also examined by recombination PCR detection to confirm the specific deletion of Col1a1 by gene recombination shown by the expected lanes specifically in myofibroblasts from KPPF;Col1^{fspKO} mice. FIG. 14D. Serial sections of pancreatic tumor sections from KPPC and KPPC;Col1^{fspKO} mice, stained with hematoxylin and eosin (H&E), Col1 (immunohistochemistry), and Picrosirius Red.

FIGS. 15A-C. FIG. 15A. Genetic strategy to induce EGFP expression in Pdx1-Flp lineage and tdTomato expression in Fsp1-Cre lineage using the R26^{Dual} tracer in KPPF;Fsp1-Cre;R26^{Dual} mice. FIG. 15B. Representative images of Fsp1-induced intrinsic tdTomato and αSMA immunofluorescence staining among fibroblasts of primary tumors from KPPF;Fsp1-Cre;R26^{Dual} mice. FIG. 15C. Representative images of Fsp1 and αSMA immunofluorescence stainings of primary tumors from KPPF;Cre-negative;R26^{Dual} mice (which have EGFP expression in Pdx-Flp lineage cancer cells but no tdTomato expression).

### DETAILED DESCRIPTION

Tumors contain both cancer cells and constituents of the tumor microenvironment (TME), such as fibroblasts and type I collagen. It is still unclear if tumor microenvironment serves as a facilitator of tumor growth or restrains tumor growth. There is a possibility that some aspects of the TME can serve as positive regulators of tumor progression and others as negative regulators of tumor growth. Type I collagen (collagen I) produced by the myofibroblasts is a heterotrimer that involves two α1 chains of collagen I (α1(I) collagen) and one α2 chain of collagen I (α2(I) collagen) that is cancer/tumor restraining via binding with potential receptors on cancer cells and other stromal cells (likely discodin domain receptor II-DDR2) and immune cells. In contrast, the cancer cells produce collagen I homotrimers with three α1(I) collagen chains that is cancer/tumor promoting and binds to specific receptors in cancer cells such as discodin domain receptor 1 (DDR1) to induce pro-survival signals, anti-apoptotic signals, proliferation signals, and pro-oncogenic signals. The homotrimers (made by cancer cells) are resistant to metalloproteinases and other proteinases when compared to the heterotrimers made by myofibroblasts in the tumor microenvironment. The homotrimers exhibit different structures with the exposure of distinct epitopes compared to heterotrimers, and antibodies generated against the homotrimers will have tumor inhibitory properties by disrupting the signaling through pro-oncogenic receptors on the cancer cells, among other mechanisms. DDR1 blockade leading to specific inhibition of homotrimers to DDR1 leads to suppression of cancer progression and induces anti-survival, apoptotic signals, anti-proliferation signals, and anti-oncogenic signals.

Desmoplasia and prominent deposition of extracellular matrix (ECM), such as type I collagen (Col1), are the defining features of pancreatic ductal carcinoma (PDAC). However, the specific roles of Col1, one of the most abundant proteins in PDAC, still remains controversial. Here, a next-generation dual-recombinase system (DRS) was used to achieve the genetic ablation of Col1α1 specifically in myofibroblasts or cancer cells in the context of Kras^{G12D}-driven spontaneous PDAC in mice. Intriguingly, Col1 deletion in α-smooth muscle actin (αSMA)-expressing myofibroblasts resulted in accelerated PDAC progression and animal death, whereas Col1α1 deletion in Pdx1-lineage cancerous cells led to alleviated PDAC development and prolonged survival. Cancer-derived Col1 was unique homotrimer (α1)₃ in contrast to the Col1 heterotrimer (α1/α2/α1) produced by fibroblasts. These different structures of Col1 (homotrimer versus heterotrimer) resulted in distinct behaviors of cancer cells.

Current genetically engineered mouse models (GEMMs) of PDAC, such as the classic KPC (LSL-Kras^{G12D/+};Trp53^{R172H/+} or Trp53^{loxP/loxP};Pdx1-Cre) model, have provided valuable platforms mimicking the clinical situation of human PDAC, and have enormously contributed to the research on PDAC and its therapeutics (Hingorani et al., 2005). The conventional KPC models have been extensively used in combination with genetic ablation of floxed (flanked by loxP sites) genes in cancer cells (using the same pancreatic-specific Cre, such as Pdx1-Cre or P48-Cre), or with whole-body knockout (KO) of genes. However, it remains impossible to achieve cell-type-specific genetic manipulations in stromal cell subpopulations (such as myofibroblasts or immune cells) in these GEMMs, due to the universal Cre-loxP recombination mechanism. And it is also impossible to establish KPC models containing the whole-body KO of those genes with KO lethality, for instance, Col1a1 that encodes type I collagen α1 chain (Lohler et al., 1984). Thus, there is no PDAC GEMM that enables the functional KO of Col1 in stromal cell source(s) to test the origin and contribution of Col1, especially considering Col1 is an essential component of and most abundant protein in the PDAC desmoplasia and microenvironment.

To overcome such limitations in PDAC GEMMs, a next-generation dual-recombinase system, integrating both the Cre-loxP and Flp-FRT systems, has been recently developed (Schonhuber et al., 2014). For the first time, this DRS allows for the deletion of Col1 in PDAC in order to functionally elucidate the specific roles of Col1 produced by specific cell populations, e.g., myofibroblasts or cancer cells, in the context of oncogenic Kras-induced PDAC.

Col1α1 was selected as the target for the genetic ablation of Col1 due to the fact that Col1α1 is essential for all Col1 fibers since Col1α2 alone cannot generate any form of Col1 fiber (regardless of homotrimer or heterotrimer). The DRS was used to achieve the genetic ablation of Col1 specifically in αSMA-lineage activated PSCs (FSF-Kras^{G12D/+};Pdx1-Flp;αSMA-Cre;Col1a1^{loxP/loxP}) or in Pdx1-lineage cancer cells (FSF-Kras^{G12D/+};Pdx1-Flp;Pdx1-Cre;Col1a1^{loxP/loxP}). In parallel, Col1 was depleted in αSMA-lineage activated PSCs using a more acute DRS model harboring homozygous p53 loss (FSF-Kras^{G12D/+};Trp53^{frt/frt};Pdx1-Flp;αSMA-Cre;Col1a1^{loxP/loxP}) and in Pdx1-lineage cancer cells using the conventional Cre-loxP-based KPC model (LSL-Kras^{G12D/+};Trp53^{loxP/loxP};Pdx1-Cre;Col1a1^{loxP/loxP}) also with homozygous p53 loss. By directly comparing the phenotypes of these PDAC GEMMs, the cellular sources and distinct contributions of Col1 in PDAC microenvironment were ascertained. PanIN/PDAC development was accelerated by the genetic ablation of Col1 in αSMA-lineage activated PSCs but was delayed by Col1 ablation in Pdx1-lineage cancer cells. These results underscore the tumor-restraining function of activated PSC-derived Col1, as well as the tumor-protective function of cancer cell-derived Col1.

Using pancreatic cancer as an example, it was shown that the pathogenic collagen is produced by cancer cells and not the myofibroblasts. The collagen I made is cancer cells is variant called the α1 homotrimer, while the collagen I made by myofibroblasts is non-pathogenic and helps restrain PDAC and is a α1/α2/α1 heterotrimer. The homotrimer is resistant to proteases and enzymes and remains around cancer cells aiding in their growth and invasion. The homotrimers engage receptors, such as DDR1, to induce pro-survival and pro-oncogenic signals. Inhibition of DDR1 and homotrimer formation or its ability to induce pro-oncogenic signals by small molecules or antibodies leads to control of PDAC and suppression of tumor growth. Disruption of chaperones in the cancer cells that disrupt the formation of homotrimers will also control PDAC. Inhibition of collagen 1 crosslinking enzymes specific to homotrimers can be used to control tumor growth. The generation of α1(I) collagen homotrimer-specific CAR-T constructs in autologous T cells or autologous or allogeneic NK cells as immunotherapy approach will lead to eradication of early and late pancreatic tumors. The generation of bispecific antibodies that targets α1(I) collagen homotrimer via one arm and CD3 via the other arm will lead to immune-targeting by T cells to kill the cancer cells.

### I. ANTIBODIES AND PRODUCTION THEREOF

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four CH domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in CH sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the VH when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (12) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

It will be understood that monoclonal antibodies binding to homotrimeric type I collagen will have several applications. These include the production of diagnostic kits for use in detecting and diagnosing cancer, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce cancer-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art. HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described, and those using polyethylene glycol (PEG), such as 37% (v/v) PEG. The use of electrically induced fusion methods also is appropriate and there are processes for better efficiency. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Here, the preferred epitope is a conformational epitope that is present in homotrimeric type I collagen but absent in heterotrimeric type I collagen.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for treatment of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor, BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described. High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life, including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T256E.

Additionally, various publications describe methods for obtaining physiologically active molecules whose half-lives are modified either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH₂ domain (according to the IMGT unique numbering for C-domain) were substituted with alanine residues. This modification, also known as “LALA” mutation, abolishes antibody binding to FcγRI, FcγRII and FcγRIIIa. The variant and unmodified recombinant mAbs were compared for their capacity to neutralize and enhance infection by the four dengue virus serotypes. LALA variants retained the same neutralizing activity as unmodified mAb, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication US 2003/0003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection; however it has become clear that many human naturally occurring antibodies with autoreactive properties persist in adult mature repertoires. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′).sub.2 bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

F. Chimeric Antigen Receptors

Chimeric antigen receptor molecules are recombinant fusion protein and are distinguished by their ability to both bind antigen and transduce activation signals via immunoreceptor activation motifs (ITAMs) present in their cytoplasmic tails. Receptor constructs utilizing an antigen-binding moiety (for example, generated from single chain antibodies (scFv) afford the additional advantage of being “universal” in that they bind native antigen on the target cell surface in an HLA-independent fashion.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

Embodiments of the CARs described herein include nucleic acids encoding an antigen-specific chimeric antigen receptor (CAR) polypeptide, including a comprising an intracellular signaling domain, a transmembrane domain, and an extracellular domain comprising one or more signaling motifs. In certain embodiments, the CAR may recognize an epitope comprised of the shared space between one or more antigens. In some embodiments, the chimeric antigen receptor comprises: a) an intracellular signaling domain, b) a transmembrane domain, and c) an extracellular domain comprising an antigen binding domain. Optionally, a CAR can comprise a hinge domain positioned between the transmembrane domain and the antigen binding domain. In certain aspects, a CAR of the embodiments further comprises a signal peptide that directs expression of the CAR to the cell surface. For example, in some aspects, a CAR can comprise a signal peptide from GM-CSF.

In certain embodiments, the CAR can also be co-expressed with a membrane-bound cytokine to improve persistence when there is a low amount of tumor-associated antigen. For example, CAR can be co-expressed with membrane-bound IL-15.

Depending on the arrangement of the domains of the CAR and the specific sequences used in the domains, immune effector cells expressing the CAR may have different levels activity against target cells. In some aspects, different CAR sequences may be introduced into immune effector cells to generate engineered cells, the engineered cells selected for elevated SRC and the selected cells tested for activity to identify the CAR constructs predicted to have the greatest therapeutic efficacy.

1. Antigen Binding Domain

In certain embodiments, an antigen binding domain can comprise complementary determining regions of a monoclonal antibody, variable regions of a monoclonal antibody, and/or antigen binding fragments thereof. In another embodiment, that specificity is derived from a peptide (e.g., cytokine) that binds to a receptor. A “complementarity determining region (CDR)” is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen—each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions.

It is contemplated that the CAR nucleic acids, in particular the scFv sequences are human genes to enhance cellular immunotherapy for human patients. In a specific embodiment, there is provided a full length CAR cDNA or coding region. The antigen binding regions or domains can comprise a fragment of the VH and VL chains of a single-chain variable fragment (scFv) derived from a particular mouse, or human or humanized monoclonal antibody. The fragment can also be any number of different antigen binding domains of an antigen-specific antibody. In a more specific embodiment, the fragment is an antigen-specific scFv encoded by a sequence that is optimized for human codon usage for expression in human cells. In certain aspects, VH and VL domains of a CAR are separated by a linker sequence, such as a Whitlow linker. CAR constructs that may be modified or used according to the embodiments are also provided in International (PCT) Patent Publication No. WO/2015/123642, incorporated herein by reference.

As previously described, the prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to an antigen of interest, such as tumor associated antigen. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, it is shown that a CAR may be engineered using VH and VL chains derived from different mAbs to generate a panel of CAR+ T cells. The antigen binding domain of a CAR can contain any combination of the LCDR1-3 sequences of a first antibody and the HCDR1-3 sequences of a second antibody.

2. Hinge Domain

In certain aspects, a CAR polypeptide of the embodiments can include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-gene modified T cells. In some aspects, the hinge domain comprises a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor. In certain aspects, the hinge domain (and/or the CAR) does not comprise a wild type human IgG4 CH2 and CH3 sequence.

In some cases the CAR hinge domain could be derived from human immunoglobulin (Ig) constant region or a portion thereof including the Ig hinge, or from human CD8 α transmembrane domain and CD8a-hinge region. In one aspect, the CAR hinge domain can comprise a hinge-CH₂—CH₃ region of antibody isotype IgG₄. In some aspects, point mutations could be introduced in antibody heavy chain CH₂ domain to reduce glycosylation and non-specific Fc gamma receptor binding of CAR-T cells or any other CAR-modified cells.

In certain aspects, a CAR hinge domain of the embodiments comprises an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. In some aspects, a CAR hinge domain comprises an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. In further aspects, a CAR hinge domain can comprise an IgG4-Fc domain having an amino acid substitution at position L235 for an amino acid that is hydrophilic, such as R, H, K, D, E, S, T, N or Q or that has similar properties to an “E” such as D. In certain aspects, a CAR hinge domain can comprise an IgG4-Fc domain having an amino acid substitution at position N297 for an amino acid that has similar properties to a “Q” such as S or T.

In certain specific aspects, the hinge domain comprises a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain or an engineered hinge domain.

3. Transmembrane Domain

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD3ζ domain, or a cysteine mutated human CD3ζ domain, or other transmembrane domains from other human transmembrane signaling proteins, such as CD16 and CD8 and erythropoietin receptor. In some aspects, for example, the transmembrane domain comprises a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions of particular use in this invention may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

4. Intracellular Signaling Domain

The intracellular signaling domain of the chimeric antigen receptor of the embodiments is responsible for activation of at least one of the normal effector functions of the immune cell engineered to express a chimeric antigen receptor. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. In some aspects, the intracellular signaling domain is derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. In a preferred embodiment, the human CD3ζ intracellular domain is used as the intracellular signaling domain for a CAR of the embodiments.

In specific embodiments, intracellular receptor signaling domains in the CAR include those of the T cell antigen receptor complex, such as the ζ chain of CD3, also Fcγ RIII costimulatory signaling domains, CD28, CD27, DAP10, CD137, OX40, CD2, alone or in a series with CD3ζ, for example. In specific embodiments, the intracellular domain (which may be referred to as the cytoplasmic domain) comprises part or all of one or more of TCRζ chain, CD28, CD27, OX40/CD134, 4-1BB/CD137, FcεRIγ, ICOS/CD278, IL-2β/CD122, IL-2Rα/CD132, DAP10, DAP12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example the CD28 and 4-1BB can be combined in a CAR construct.

In some embodiments, the CAR comprises additional other costimulatory domains. Other costimulatory domains can include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

G. ADCs

Antibody Drug Conjugates or ADCs are a new class of highly potent biopharmaceutical drugs designed as a targeted therapy for the treatment of people with disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

I. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. One means of delivery comprises the use of lipid-based nanoparticles, or exosomes, as taught in U.S. Pat. Appln. Publn. 2018/0177727, which is incorporated by reference here in its entirety. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

By virtue of their ability to enter cells, intrabodies have additional uses that other types of antibodies may not achieve. In the case of the present antibodies, the ability to interact with the DDR1 cytoplasmic domain in a living cell may interfere with functions associated with the DDR1, such as signaling functions (binding to other molecules) or oligomer formation. In particular, it is contemplated that such antibodies can be used to inhibit type I collagen homotrimer formation by, for example, disrupting associated chaperone or crosslinking enzyme functions.

J. Purification

The antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

K. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### II. METHODS OF TREATMENT

Certain aspects of the present embodiments can be used to prevent or treat a disease or disorder associated with the presence of homotrimeric type I collagen, such as pancreatic ductal adenocarcinoma. Functioning of homotrimeric type I collagen may be reduced by any suitable drugs. Preferably, such substances would be an anti-homotrimeric type I collagen antibody.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that inhibits homotrimeric type I collagen either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease, such as a cancer or a fibroid disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor, meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular, mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

B. Formulation and Administration

The present disclosure provides pharmaceutical compositions comprising antibodies that selectively bind to homotrimeric type I collagen. Such compositions comprise a prophylactically or therapeutically effective amount of an antibody or a fragment thereof and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies generally will involve the use of intravenous or intramuscular injections. The forms of antibody can be as monoclonal antibodies (MAb). Such immunity generally lasts for only a short period of time, and there is also a potential risk for hypersensitivity reactions, and serum sickness, especially from gamma globulin of non-human origin. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

C. Kits and Diagnostics

In various aspects of the embodiments, a kit is envisioned containing therapeutic agents and/or other therapeutic and delivery agents. The present embodiments contemplate a kit for preparing and/or administering a therapy of the embodiments. The kit may comprise one or more sealed vials containing any of the pharmaceutical compositions of the present embodiments. The kit may include, for example, at least one homotrimeric type I collagen antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

D. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

E. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have a cytotoxic effect.

F. Combination Therapy

In certain embodiments, the compositions and methods of the present embodiments involve an antibody or an antibody fragment against homotrimeric type I collagen to inhibit its activity, in combination with a second or additional therapy, such as chemotherapy or immunotherapy. Such therapy can be applied in the treatment of any disease that is associated with elevated homotrimeric type I collagen. For example, the disease may be a cancer or a fibroid disease.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

A therapeutic antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169); cytokine therapy, e.g., interferons α, α, and γ, IL-1, GM-CSF, and TNF; gene therapy, e.g., TNF, IL-1, IL-2, and p53 (U.S. Pat. Nos. 5,830,880 and 5,846,945); and monoclonal antibodies, e.g., anti-CD20, anti-ganglioside GM2, and anti-p185 (U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, may be antibodies, such as human antibodies (e.g., International Patent Publication WO2015016718, incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. IMMUNODETECTION METHODS

In still further embodiments, the present disclosure concerns immunodetection methods for binding, quantifying, and otherwise generally detecting homotrimeric type I collagen. Other immunodetection methods include specific assays for determining the presence of homotrimeric type I collagen in a subject. A wide variety of assay formats are contemplated, but specifically those that would be used to detect homotrimeric type I collagen in a tissue sample obtained from a subject, such as a biopsy. These assays may be packaged in the form of a kit with appropriate reagents and instructions to permit use.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature. In general, the immunobinding methods include obtaining a sample suspected of containing homotrimeric type I collagen and contacting the sample with a first antibody in accordance with the present disclosure, as the case may be, under conditions effective to allow the formation of immunocomplexes.

The immunobinding methods also include methods for detecting and quantifying the amount of homotrimeric type I collagen or related components in a sample and the detection and quantification of any immune complexes formed during the binding process. Here, one would obtain a sample suspected of containing homotrimeric type I collagen and contact the sample with an antibody that binds homotrimeric type I collagen, followed by detecting and quantifying the amount of immune complexes formed under the specific conditions. In terms of antigen detection, the biological sample analyzed may be any sample that is suspected of containing homotrimeric type I collagen, such as a tissue section or specimen, a homogenized tissue extract, or a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to homotrimeric type I collagen. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the homotrimeric type I collagen is added to the wells. After binding and washing to remove non-specifically bound immune complexes, the bound antigen may be detected. Detection may be achieved by the addition of another anti-homotrimeric type I collagen antibody that is linked to a detectable label. This type of ELISA is a simple “sandwich ELISA.” Detection may also be achieved by the addition of a second anti-homotrimeric type I collagen antibody, followed by the addition of a third antibody that has binding affinity for the second antibody, with the third antibody being linked to a detectable label.

In another exemplary ELISA, the samples suspected of containing the homotrimeric type I collagen are immobilized onto the well surface and then contacted with the anti-homotrimeric type I collagen antibodies of the disclosure. After binding and washing to remove non-specifically bound immune complexes, the bound anti-homotrimeric type I collagen antibodies are detected. Where the initial anti-homotrimeric type I collagen antibodies are linked to a detectable label, the immune complexes may be detected directly. Again, the immune complexes may be detected using a second antibody that has binding affinity for the first anti-homotrimeric type I collagen antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of norovirus antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third “capture” molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art.

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

E. Immunodetection Kits

In still further embodiments, the present disclosure concerns immunodetection kits for use with the immunodetection methods described above. As the antibodies may be used to detect homotrimeric type I collagen, the antibodies may be included in the kit. The immunodetection kits will thus comprise, in suitable container means, a first antibody that binds to homotrimeric type I collagen, and optionally an immunodetection reagent.

In certain embodiments, the homotrimeric type I collagen antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of homotrimeric type I collagen, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

Mice. FSF-Kras^{G12D/+} (Schonhuber et al., 2014), Pdx1-Flp (Schonhuber et al., 2014), Trp53^{frt/+} (Lee et al., 2012), LSL-Kras^{G12D/+} (Hingorani et al., 2005), Trp53^{loxP/+} (Chen et al., 2005), Pdx1-Cre (Hingorani et al., 2005), αSMA-Cre (LeBleu et al., 2013), and Fsp1-Cre (Xue et al., 2003; Bhowmick et al., 2004) mouse strains were previously documented. Col1a1^{loxP/loxP} mouse strain (with loxP-flanked exons 2-5) was established from the Col1a1^{tm1a(EUCOMM)Wtsi} strain that was purchased from European Mouse Mutant Cell Repository (EuMMCR). The Rosa26-CAG-loxP-frt-Stop-frt-FirefyLuc-EGFP-loxP-RenillaLuc-tdTomato (referred to as R26^{Dual}) mouse strain contains the novel R26^{Dual} dual-fluorescence reporter allele, which allows for EGFP expression under the control of the Pdx1-Flp transgene, or for tdTomato expression under the control of the αSMA-Cre and Fsp1-Cre transgenes. Characterization of genotyping and disease phenotypes for the FSF-Kras^{G12D/+};Pdx1-Flp (referred to as KF) or FSF-Kras^{G12D/+};Trp53^{frt/frt};Pdx1-Flp (referred to as KPPF) mice was performed as previously described (Schonhuber et al., 2014). The KF and KPPF mice were crossed with the αSMA-Cre, Pdx1-Cre, Fsp1-Cre, Col1a1^{loxP/loxP}, or R26^{Dual} mouse strains, resulting in the generation of the KF;αSMA-Cre;Col1a1^{loxP/loxP} (referred to as KF;Col1^{smaKO}), KF;Pdx1-Cre;Col1a1^{loxP/loxP} (referred to as KF;Col1^{pdxKO}), KPPF;αSMA-Cre;Col1a1^{loxP/loxP} (referred to as KPPF;Col1^{smaKO}), and KPPF;Fsp1-Cre;Col1a1^{loxP/loxP} (referred to as KPPF;Col1^{fspKO}) mice. These mice allow for Col1a1 deletion in PDAC-associated fibroblast subpopulations expressing αSMA or Fsp1. The LSL-Kras^{G12D};Pdx1-Cre (referred to as KC) or LSL-Kras^{G12D};Trp53^{loxP/loxP};Pdx1-Cre (referred to as KPPC) mice were crossed with the Col1a1^{loxP/loxP} mouse strain, resulting in the generation of the KC;Col1a1^{loxP/loxP} (referred to as KC;Col1^{pdxKO}) and KPPC;Col1a1^{loxP/loxP} (referred to as KPPC;Col1^{pdxKO}) mice. These mice allow for Col1a1 deletion in PDAC cells. The aforementioned experimental mice with desired genotypes were monitored and analyzed with no randomization or blinding. Both female and male mice with desired genotype(s) for PDAC were used for experimental mice. All mice were housed under standard housing conditions at MD Anderson Cancer Center (MDACC) animal facilities, and all animal procedures were reviewed and approved by the MDACC Institutional Animal Care and Use Committee.

### Example 1—Dual-Recombinase System (DRS) Mouse Model Induces Spontaneous Pancreatic Cancer and Allows Genetic Modulations in Various Targeted Cell Populations

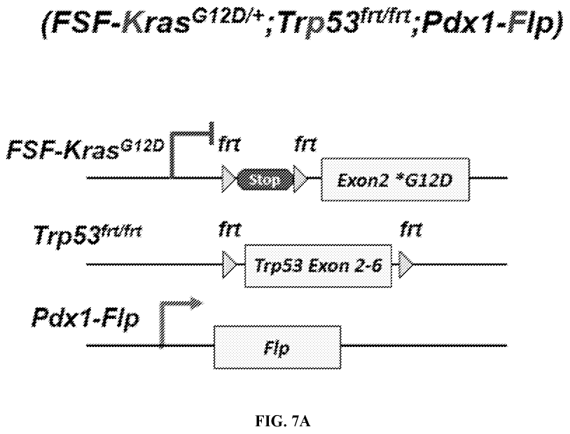

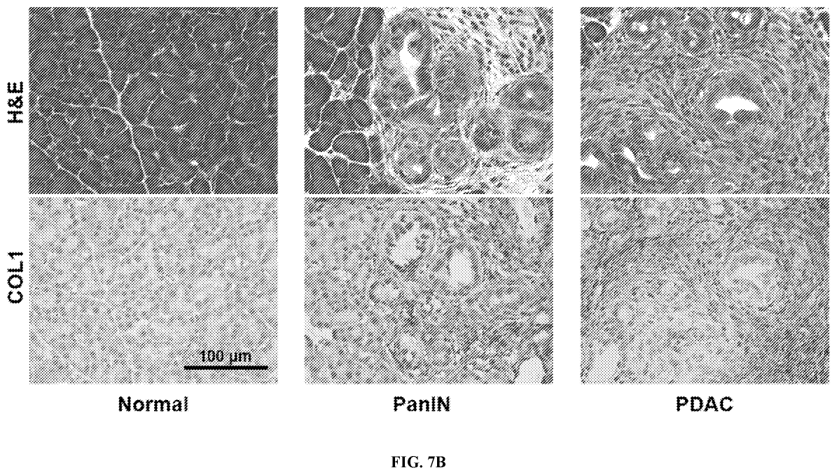

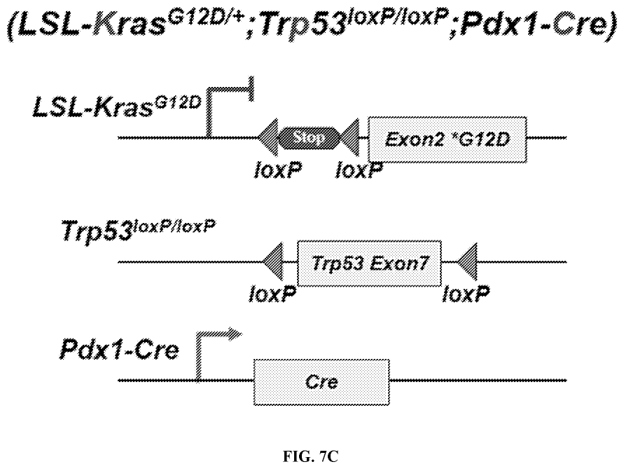

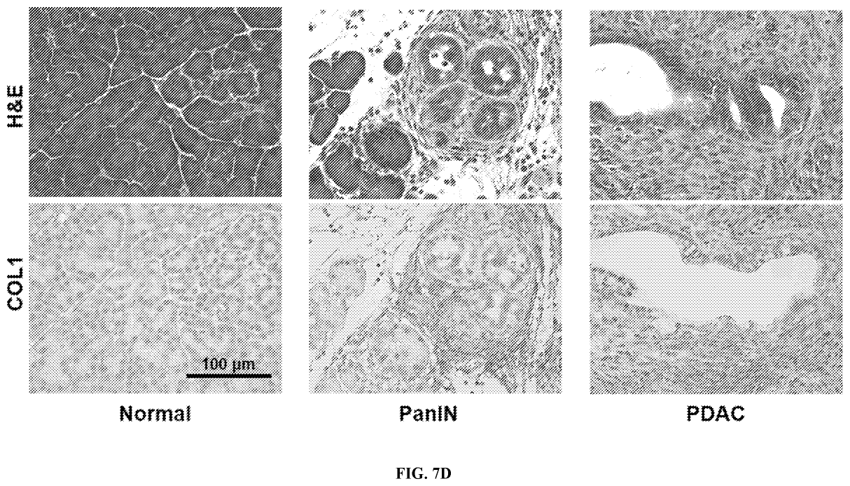

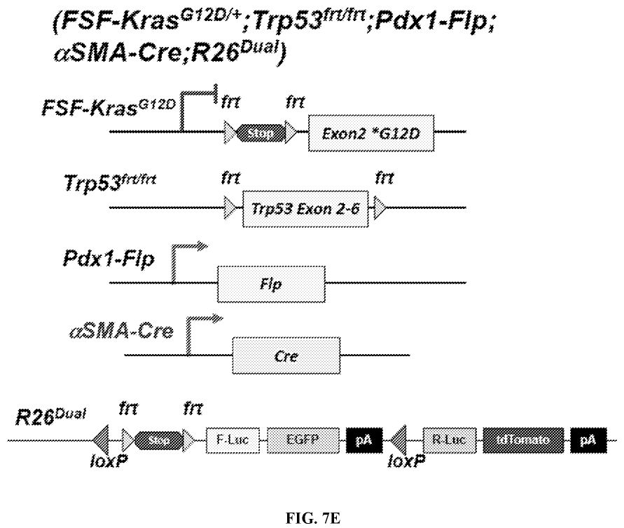

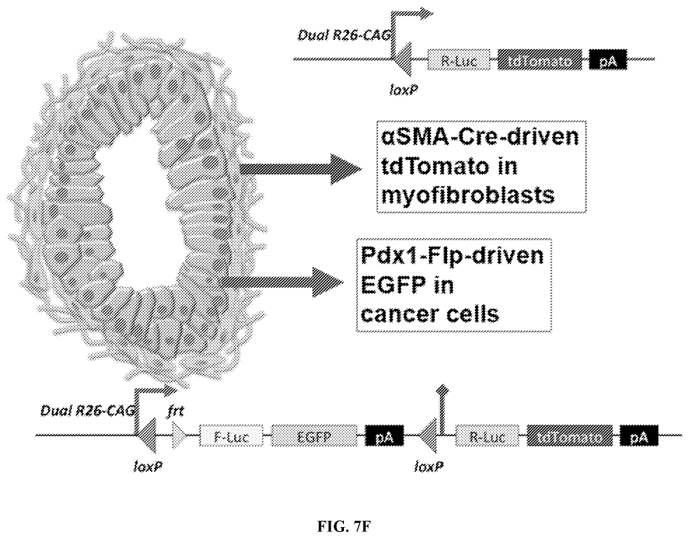

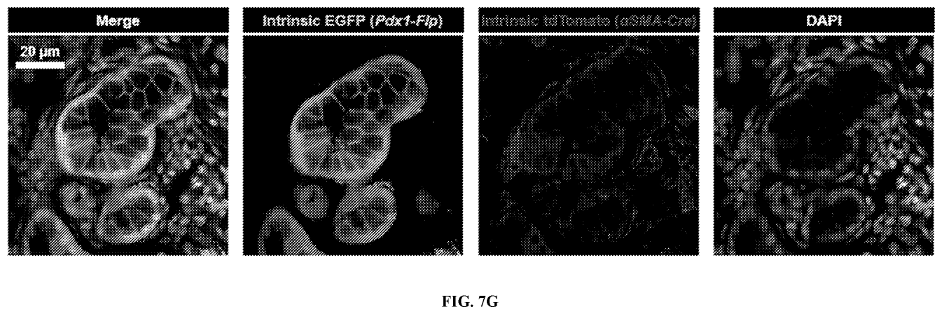

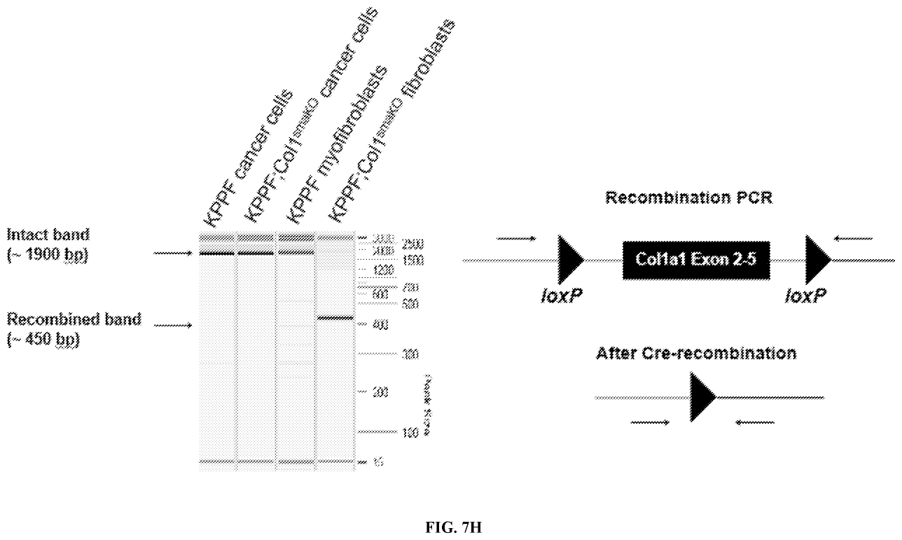

The novel dual-recombinase system (DRS) mouse model for pancreatic cancer utilized the Flippase-FRT (Flp-FRT) system to induce oncogenic Kras expression and p53 loss in Pdx1 lineage (FSF-Kras^{G12D/+};Trp53^{frt/frt};Pdx1-Flp), replacing the traditional Cre-loxP system in widely-used KPC (LSL-Kras^{G12D/+};Trp53^{R172H/+} or Trp53^{loxP/loxP};Pdx1-Cre) mouse models. This Flp-FRT-based DRS (FSF-Kras^{G12D/+};Trp53^{frt/frt};Pdx1-Flp, “KPPF” for short) mouse model develops pancreatic intraepithelial neoplasia (PanIN) and pancreatic ductal adenocarcinoma (PDAC) in an almost identical manner to the traditional Cre-loxP-based (LSL-Kras^{G12D/+};Trp53^{loxP/loxP};Pdx1-Cre, “KPPC” for short) mouse model (FIGS. 7A-D), as also documented by the original study of DRS model (Schonhuber et al., 2014). As expected, both pancreatic cancer mouse model systems exhibited prominent type I collagen (Col1) deposition during disease development. Importantly, this new DRS model system allows the addition of another genetic manipulating system with Cre transgene and floxed (flanked by loxP sites) alleles, independent of the spontaneous PDAC induced by the Flp-FRT-based system.

In order to test the functionality of this DRS mouse model harboring both Cre-loxP and Flp-FRT systems, a novel lineage-tracing dual-reporter (Rosa26-CAG-loxP-frt-Stop-frt-FirefyLuc-EGFP-loxP-RenillaLuc-tdTomato, hereafter referred to as R26^{Dual}) was used to generate the KPPF;αSMA-Cre;R26^{Dual} mice (FIG. 7E). In the PDAC tissues of this mouse strain, Pdx1-lineage cancer cells exhibited EGFP expression, while αSMA-lineage activated PSCs exhibited tdTomato expression (FIGS. 7F&G), confirming the genetic recombination by Pdx-Flp and αSMA-Cre respectively.

### Example 2—Type I Collagen (Col1) Deposition Varies Along the Stages of PanIN/PDAC Development

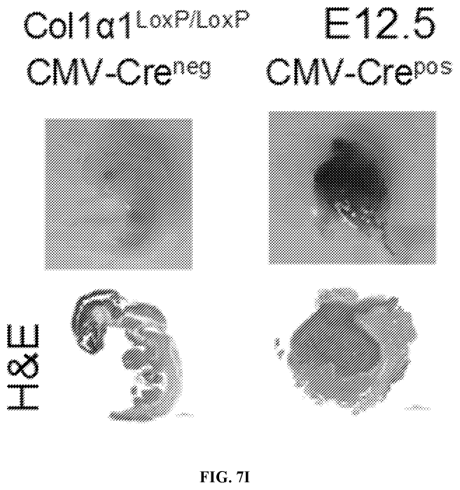

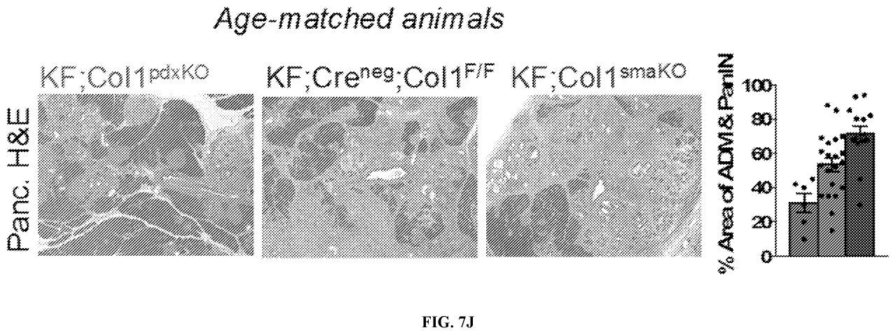

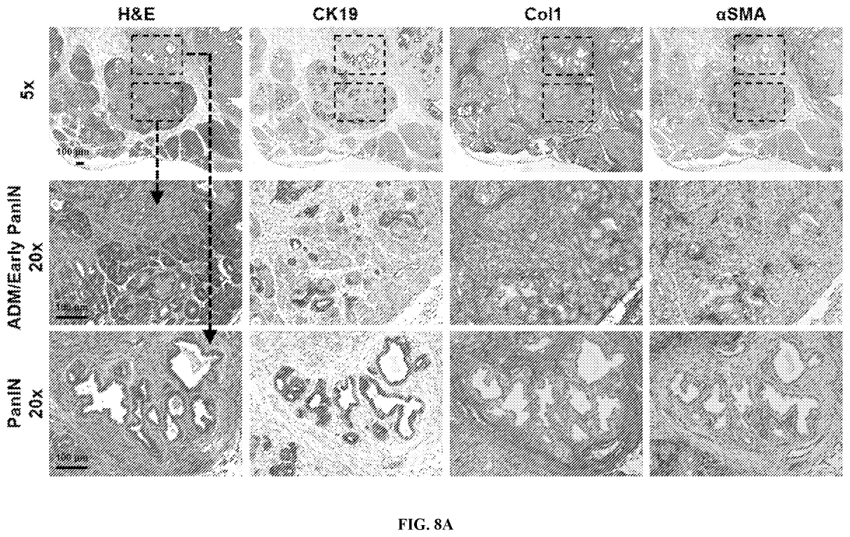

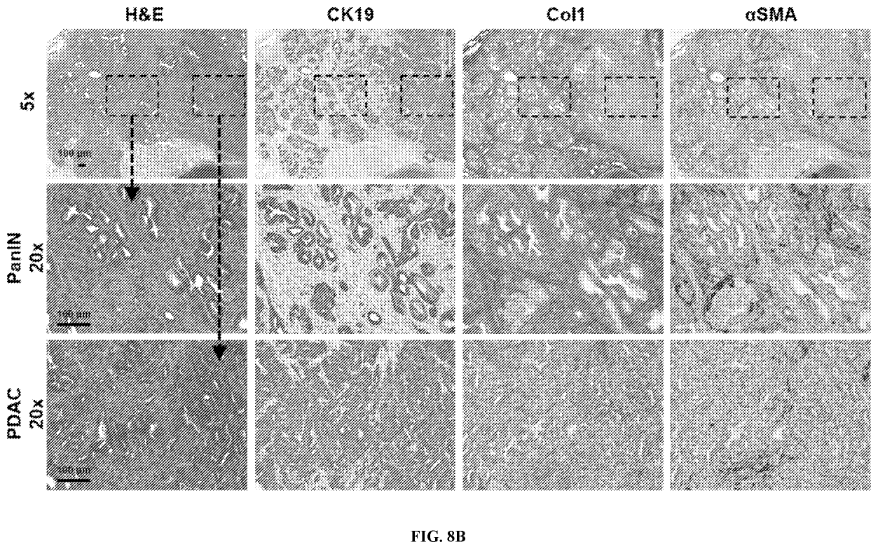

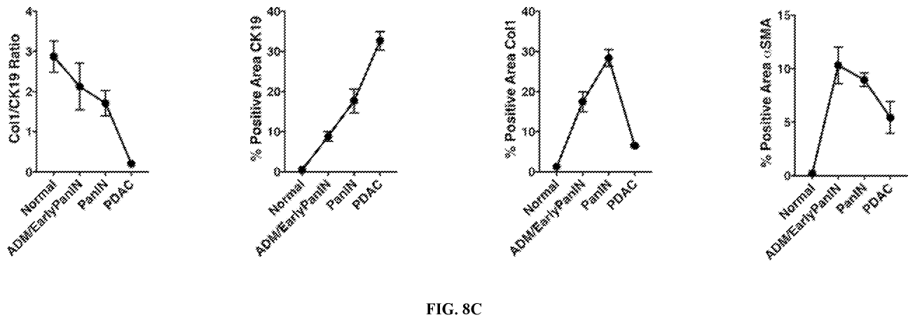

Using IHC staining methods on serial sections, the expression of Col1, in comparison to the expression levels of CK19 and αSMA (markers for cancerous cells and activated PSCs, respectively), was examined during PanIN/PDAC development (FIGS. 8A-B). The expression of the aforementioned proteins revealed dynamic changes (FIG. 8C). Normal pancreatic tissue revealed minimal/negligible presence of CK19, αSMA, or Col1. When ADM (or early PanIN) lesion emerged, αSMA immediately elevated to the highest level due to the activation of PSCs in response to pancreatic epithelial abnormality. These activated PSCs began to produce interstitial Col1, resulting in the peak level of Col1 fibers at following PanIN stages. When disease continued to develop from PanIN to PDAC, cancer cell population outgrew the stromal components, coinciding with decreased presence of αSMA- or Col1-positive areas.

Particularly, CK19 levels constantly increased throughout the PanIN/PDAC development. αSMA reached the highest level at acinar-to-ductal metaplasia (ADM) or early PanIN stage, indicating the immediate recruitment and/or activation of αSMA-positive PSCs in response to the very early phase of disease progression. In contrast, Col1 levels reached the highest level during PanIN stages and then decreased during the development into PDAC stage. PDAC tissue revealed dominant cancer cell presence (CK19-positive areas), together with diluted/decreased presence of Col1 and αSMA-positive activated PSCs.

Noticeably, despite the non-linear dynamics of Col1 level, the ratio of Col1/CK19 constantly decreased as the disease progresses. These results suggest that a decreased Col1/CK19 ratio may indicate compromised host restraints on PDAC development and a more advanced disease status.

### Example 3—Col1 Deletion in αSMA-Expressing Activated PSCs Accelerates PDAC Development and Shortens Animal Survival

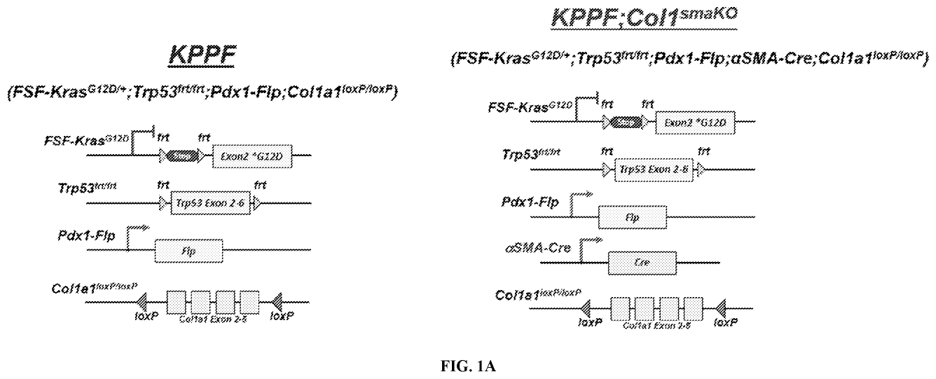

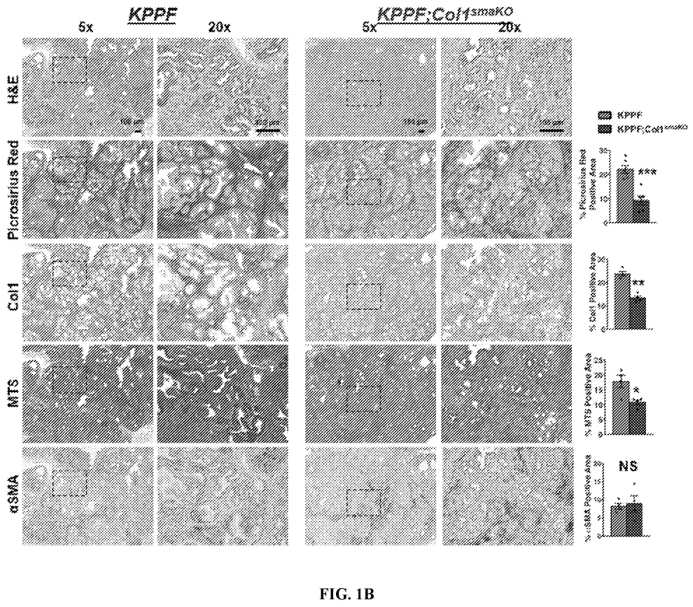

The above observations were consistent with previous studies indicating activated PSC population (rather than pancreatic cancer cells) as the major producer of Col1 in PDAC stroma. Thus, the genetic ablation of Col1 in activated PSC population using the new DRS mouse model was sought. As shown in FIG. 1A, Col1 was specifically deleted in αSMA-expressing activated PSCs of PDAC in KPPF;Col1^{smaKO} (FSF-Kras^{G12D/+};Trp53^{frt/frt};Pdx1-Flp;αSMA-Cre;Col1a1^{loxP/loxP}) mice. Col1 deletion in αSMA-expressing activated PSCs led to decreased levels of fibrillar Col1, desmoplasia, and stiffness in PDAC tissues, as shown by serial sections with IHC staining (FIG. 1B).

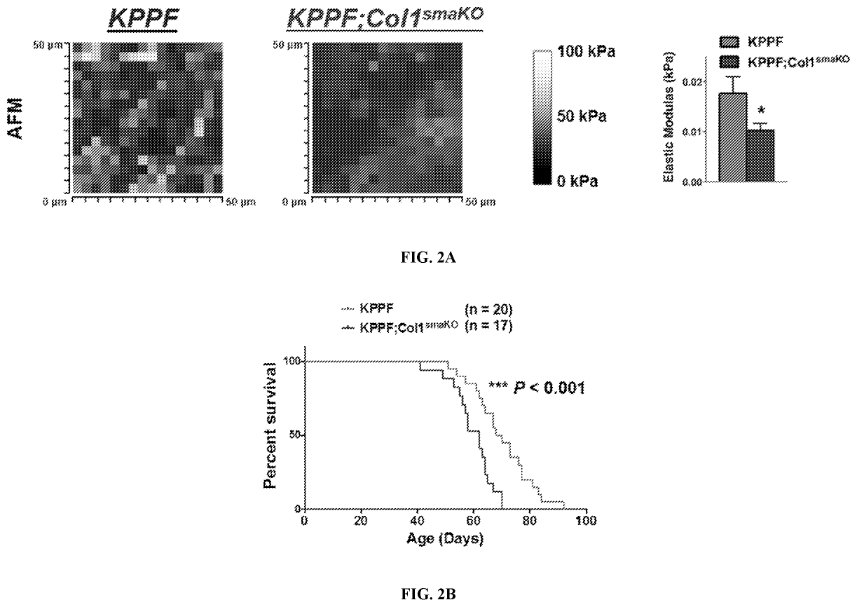

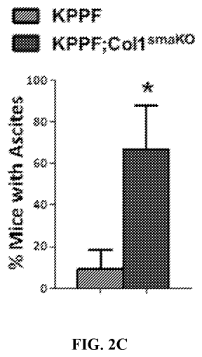

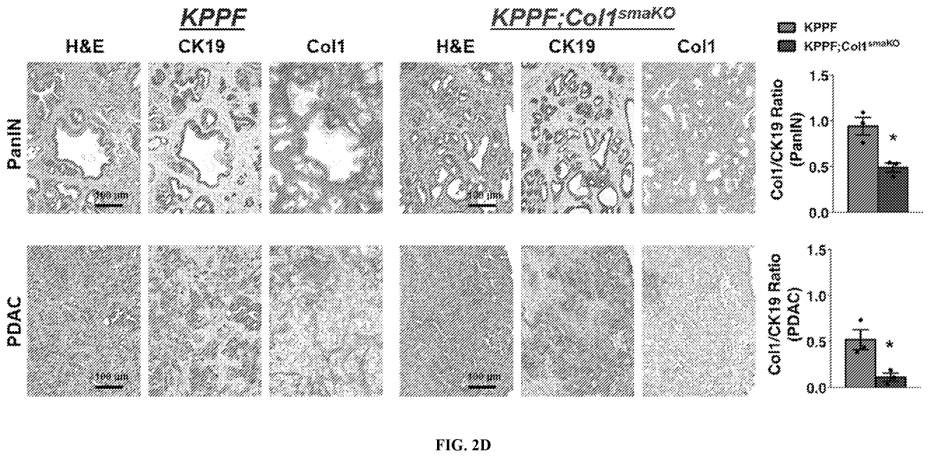

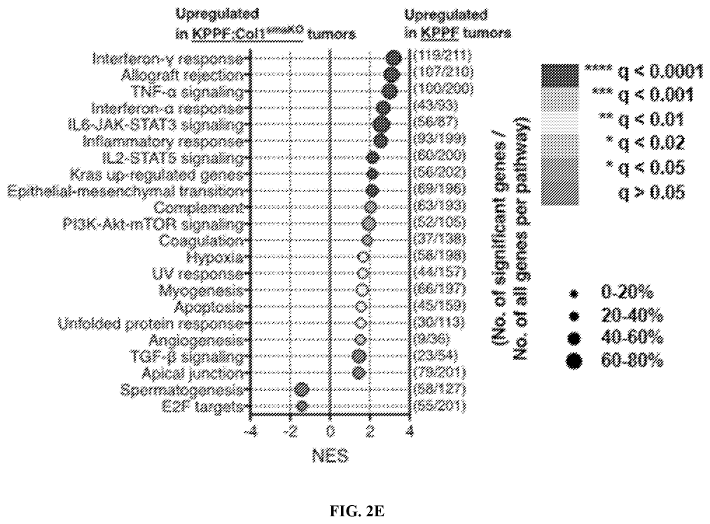

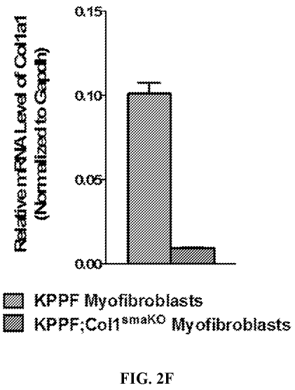

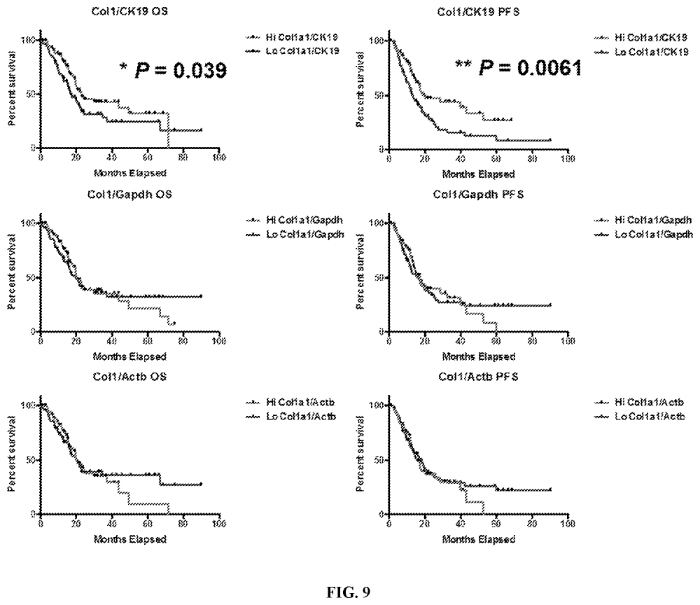

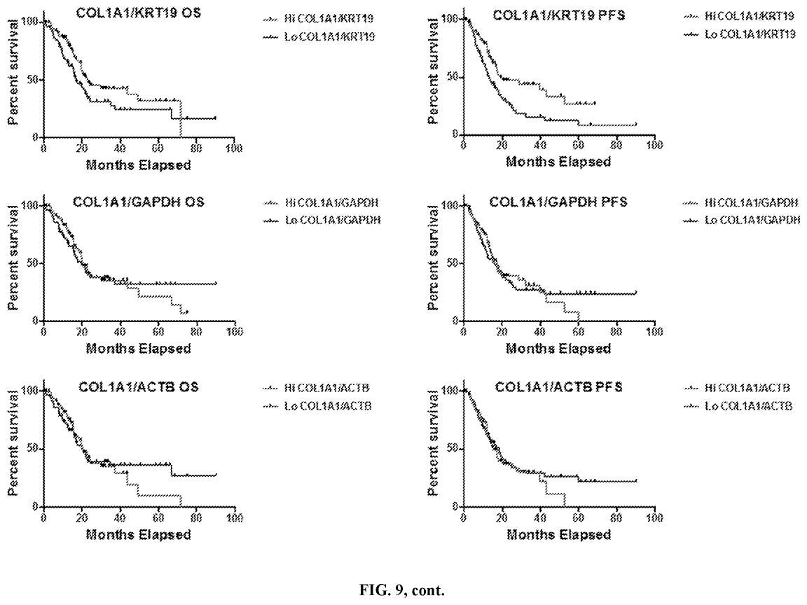

Col1 deletion in αSMA-expressing activated PSCs in the context of PDAC resulted in significantly shorter the animal survival (FIG. 2B) and higher occurrence of ascites at endpoint stage (FIG. 2C). Decreased Col1 levels in KPPF;Col1^{smaKO} tumors was observed in both PanIN and PDAC stages (FIG. 2F), which was accompanied by significantly decreased Col1/CK19 ratio in KPPF;Col1^{smaKO} tumors than in KPPF tumors (FIG. 2D). These observations further support the notion that lower Col1/CK19 ratios are correlated with compromised host restraints on PDAC development and a more advanced disease status. Next, the Col1/CK19 ratio was examined by comparing the mRNA levels of Col1a1 and CK19 (RNA Seq V2 RSEM) in human PDAC samples of TCGA database. Lower Col1/CK19 ratio was correlated with significantly worse overall survival (OS) and progression-free survival (PFS), consistent with the observations in transgenic mouse models (FIG. 9).

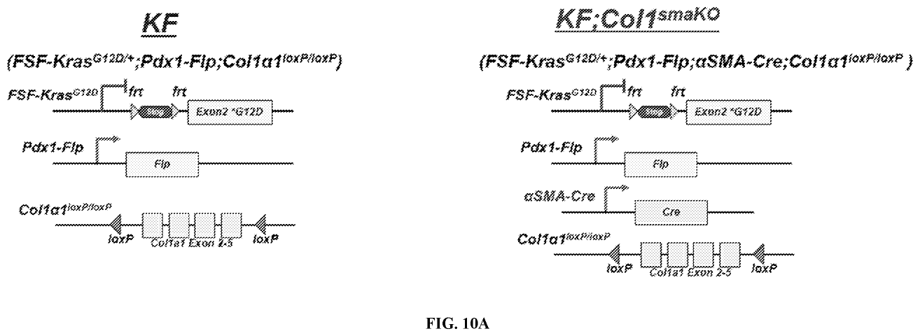

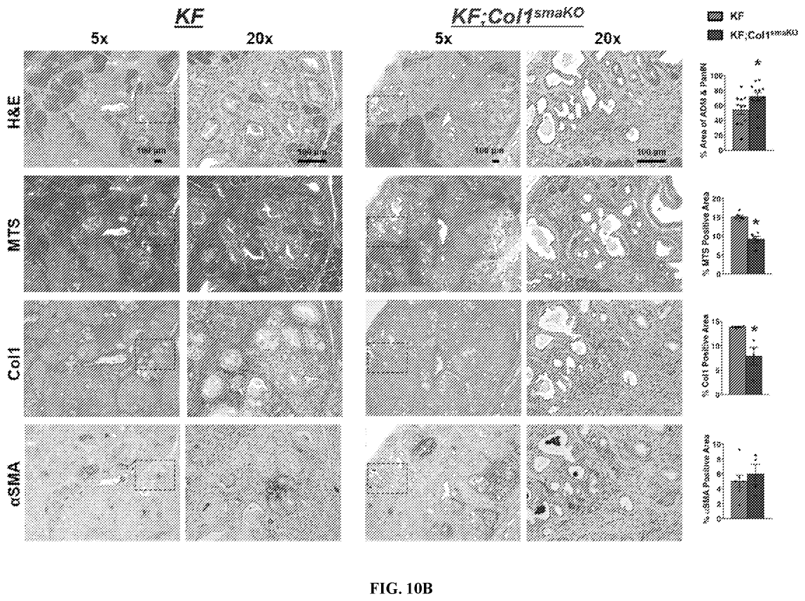

The KF;Col1^{smaKO} (FSF-Kras^{G12D/+};Pdx1-Flp;αSMA-Cre;Col1a1^{loxP/loxP}) mice, harboring oncogenic Kras mutation but not p53 loss, were generated to observe the impact of on the early stages (ADM and/or PanIN) of PDAC development (FIG. 10A). Age-matched (6-month-old) animals were examined for the occurrence of ADM and PanIN lesions. As shown in FIG. 10B, KF;Col1^{smaKO} mice exhibited significantly larger areas of ADM and PanIN lesions than KF littermate control mice. Taken together, these observations are in concordance with previous findings indicating the tumor-restraining function of myofibroblast subpopulations in the PDAC microenvironment.

### Example 4—Col1 Deletion in Pancreatic Cancer Cells Delays ADM and PanIN Development

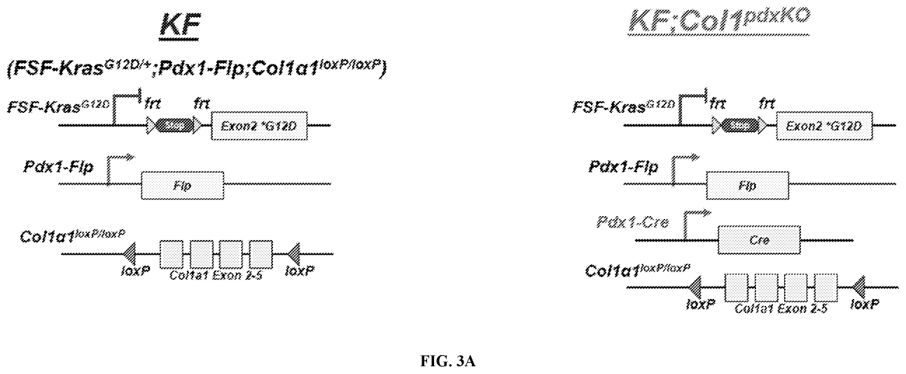

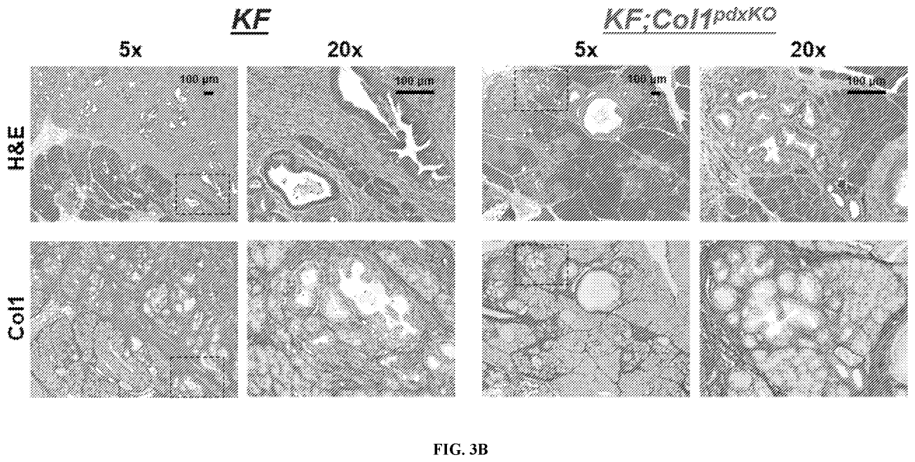

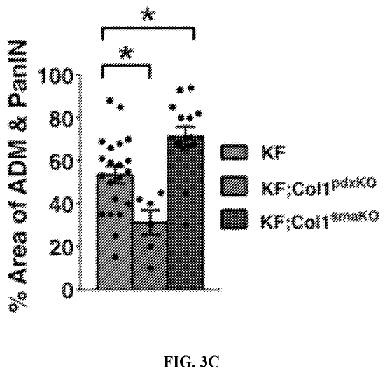

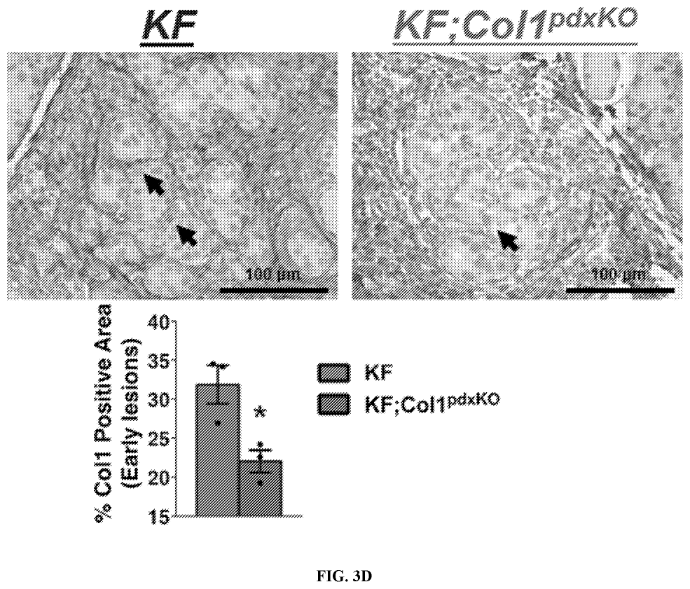

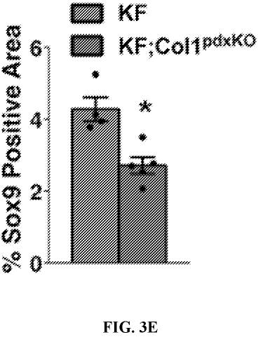

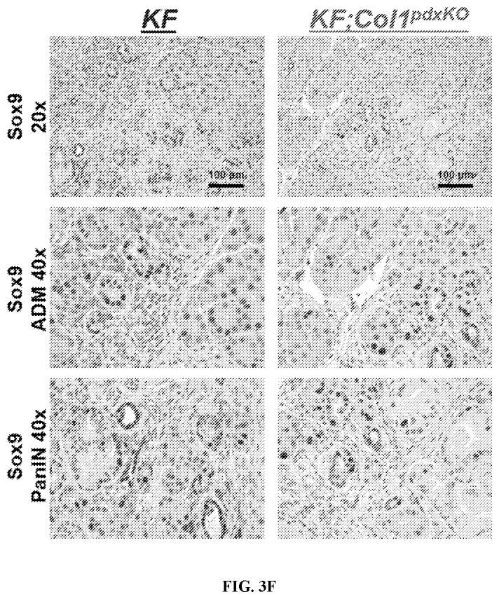

Although some studies have proposed cancer-associated fibroblasts as the major producers of Col1, other studies also emphasize the potentially unique composition and function of cancer cell-derived Col1 (Sengupta et al., 2003; Han et al., 2008; Egeblad et al., 2010; Han et al., 2010; Makareeva et al., 2010). In order to achieve genetic ablation of Col1 in cancer cells, another DRS mouse model, KF;Col1^{pdxKO} (FSF-Kras^{G12D/+};Pdx1-Flp;Pdx1-Cre;Col1a1^{loxP/loxP}), was established. The KF;Col1^{pdxKO} strain had the same KF background but integrated the Pdx1-Cre transgene (FIG. 3A) to replace the αSMA-Cre transgene of previous KF;Col1^{smaKO} strain in FIG. 10A.

Of note, the KF;Col1^{pdxKO} mouse model shared the same control mouse (KF;Cre-negative;Col1a1^{loxP/loxP}) as the KF;Col1^{smaKO} mouse, allowing for the direct comparison of disease progression status between those three strains (KF control group, KF;Col1^{smaKO} group with Col1 deletion in αSMA-expressing myofibroblasts as shown in FIG. 10A, and KF;Col1^{pdxKO} group with Col1 deletion in Pdx1-lineage cancer cells) at the same 6-month age point. Interestingly, KF;Col1^{pdxKO} mice with Col1 ablation in Pdx1-lineage cancer cells revealed significantly delayed ADM and PanIN development than KF control mice, in contrast to the accelerated disease progression in KF;Col1^{smaKO} mice (FIGS. 3B-C and 7J). Even though the pancreatic tissues of KF;Col1^{pdxKO} mice revealed significantly better histology and less ADM/PanIN areas than KF control mice, the Col1 deposition level within any given visual field of the same PanIN stage (FIG. 3B, 20× magnified panels) was not different between these two mouse groups. These results indicate that cancer cancer-derived Col1 may have important cancer-supporting function, even though its presence can be largely masked by the abundant Col1 produced by myofibroblasts at PanIN stage.

Nevertheless, a decreased Col1 level was observed in KF;Col1^{pdxKO} mice at the early stage (ADM) of disease progression, when PSC just underwent activation and had not deposited large amount of Col1 (FIG. 3D). The ADM lesions of KF;Col1^{pdxKO} mice exhibited not only reduced Col1 deposition but also significantly decreased level of Sox9 (FIGS. 3E-F), an essential marker of pancreas organogenesis as well as pancreatic cancer initiation (Seymour et al., 2007; Kopp et al., 2012). These observations indicate that Col1 deposition by cancer-initiating cells is supporting the early development of pancreatic cancer.

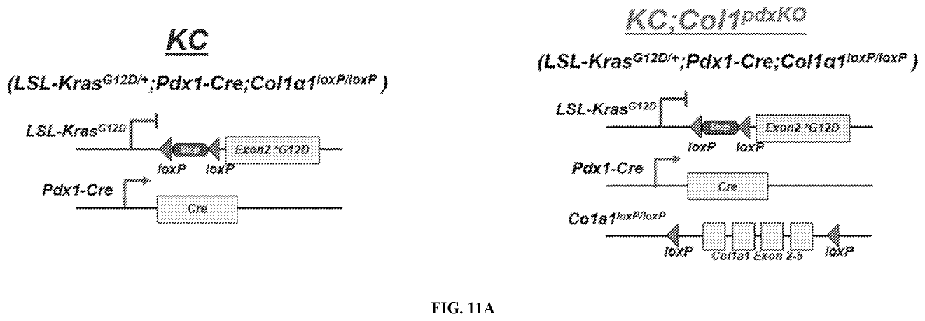

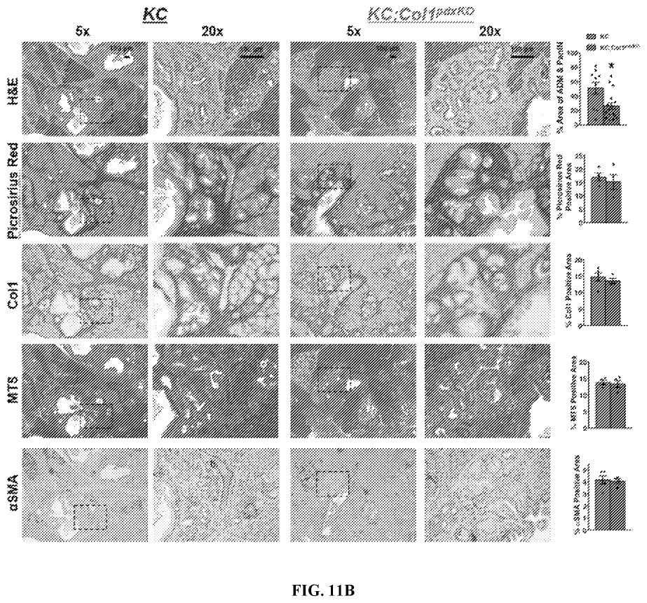

In addition to the KF;Col1^{pdxKO} mouse model, another mouse model was generated in parallel to achieve the genetic deletion of Col1 in Pdx1-lineage cancer cells. Here, the KC;Col1^{pdxKO} (SL-Kras^{G12D/+};Pdx1-Cre;Col1a1^{loxP/loxP}) mouse strain, using the conventional Cre-loxP-based system, was established in comparison with KC (LSL-Kras^{G12D/+};Pdx1-Cre) control mice (FIG. 11A). Consistent results were obtained from the KC;Col1^{pdxKO} mice, showing significantly delayed ADM and PanIN development than KC control mice at the same 6-month age point (FIG. 11B).

### Example 5—Col1 Deletion in Pancreatic Cancer Cells Delays PDAC Development and Animal Survival

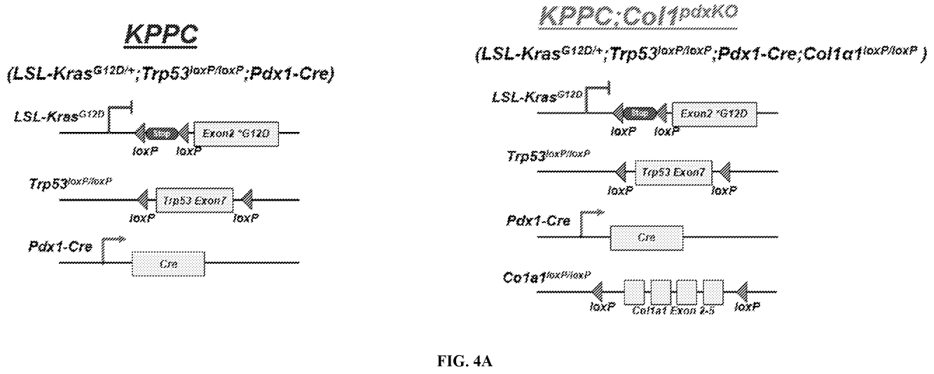

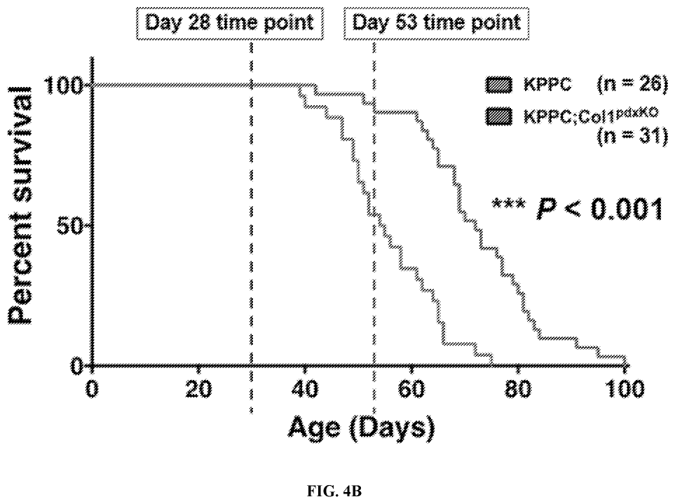

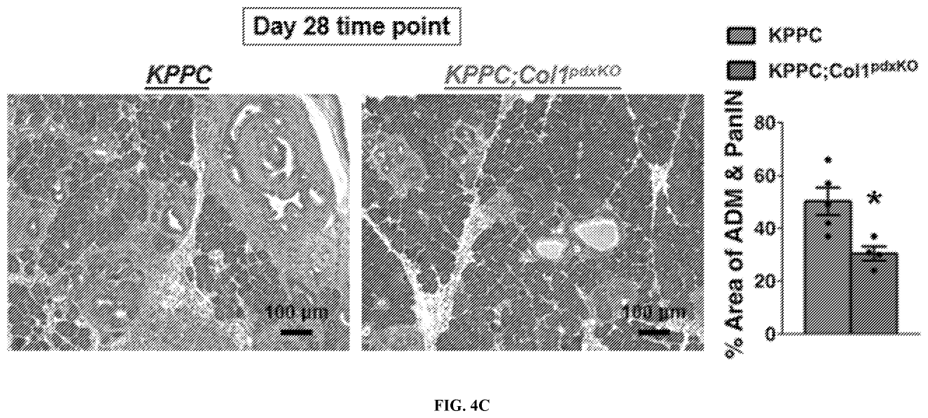

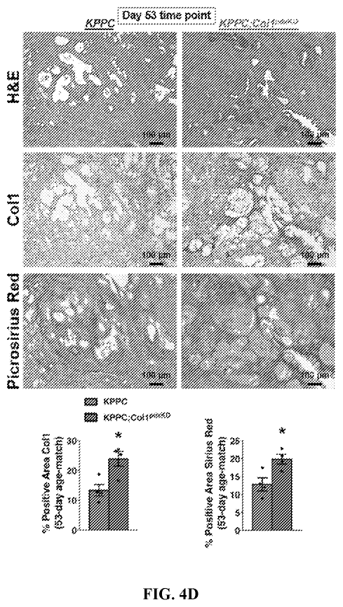

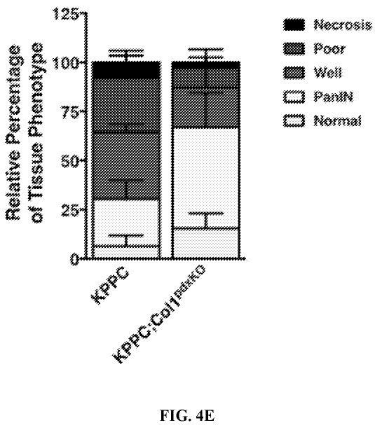

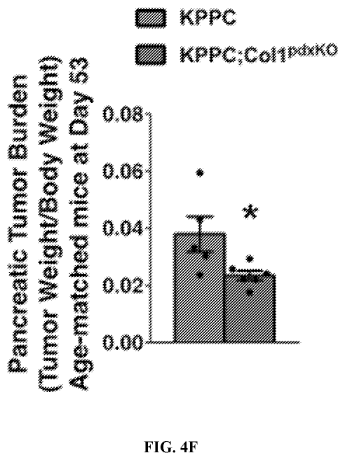

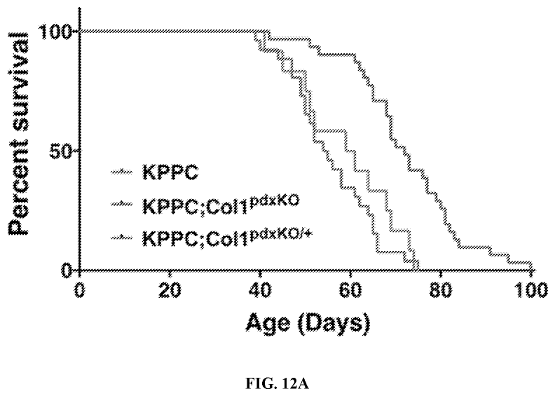

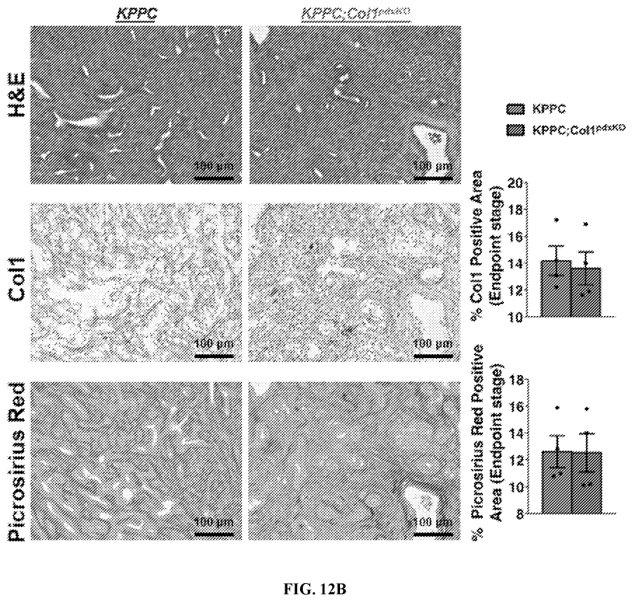

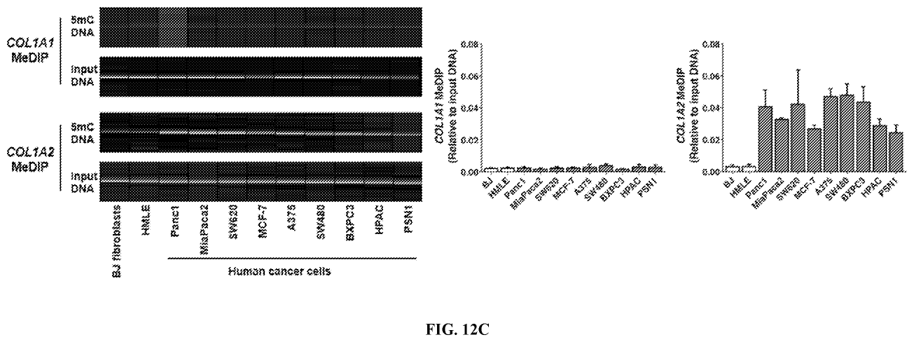

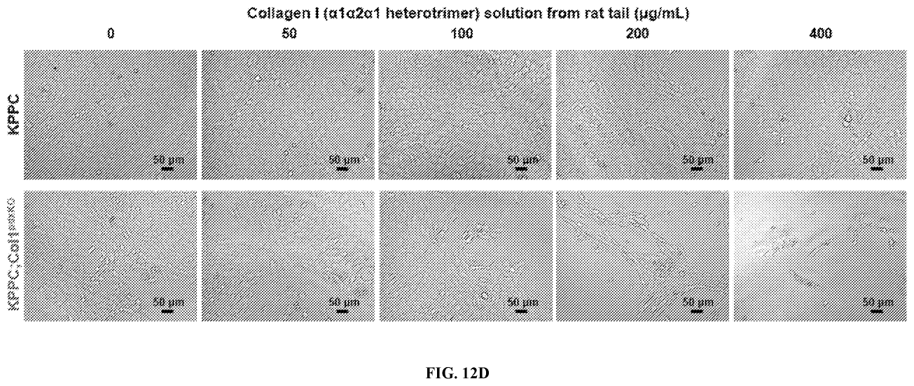

Next, a mouse model of KPPC;Col1^{pdxKO} (LSL-Kras^{G12D/+};Trp53^{loxP/loxP}; Pdx1-Cre;Col1a1^{loxP/loxP}) was generated harboring both oncogenic Kras mutation and p53 homozygous loss induced by the traditional Cre-loxP system (FIG. 4A). These mice with KPPC genetic background develop acute PDAC within 45 days, leading to animal death at the age around 55 days. Consistent with previous observations (FIGS. 3A-F and 11A-B), Col1 deletion in cancer cell lineage in KPPC;Col1^{pdxKO} mice significantly prolonged animal survival and delayed PDAC development, when compared to KPPC control mice (FIG. 4B). An additional KPPC;Col1^{pdxKO/+} strain (LSL-Kras^{G12D/+};Trp53^{loxP/loxP};Pdx1-Cre;Col1a1^{loxP/+}) with heterozygous Col1a1^{loxP} deletion in cancer cells was also generated, showing similar animal survival to that of KPPC control strain (FIG. 12A).

The early stage of disease development was examined in KPPC;Col1^{pdxKO} mice and KPPC control mice at the same age of 28 days. The pancreas of KPPC;Col1^{pdxKO} mice revealed significantly less ADM and PanIN lesions than KPPC control mice (FIG. 4C). At the same age of 52 days, KPPC;Col1^{pdxKO} (LSL-Kras^{G12D/+};Trp53^{loxP/loxP};Pdx1-Cre;Col1a1^{loxP/loxP}) revealed significantly better histology (FIGS. 4D&E) and decreased pancreatic tumor burden (FIG. 4F), as compared with age-matched KPPC control mice.

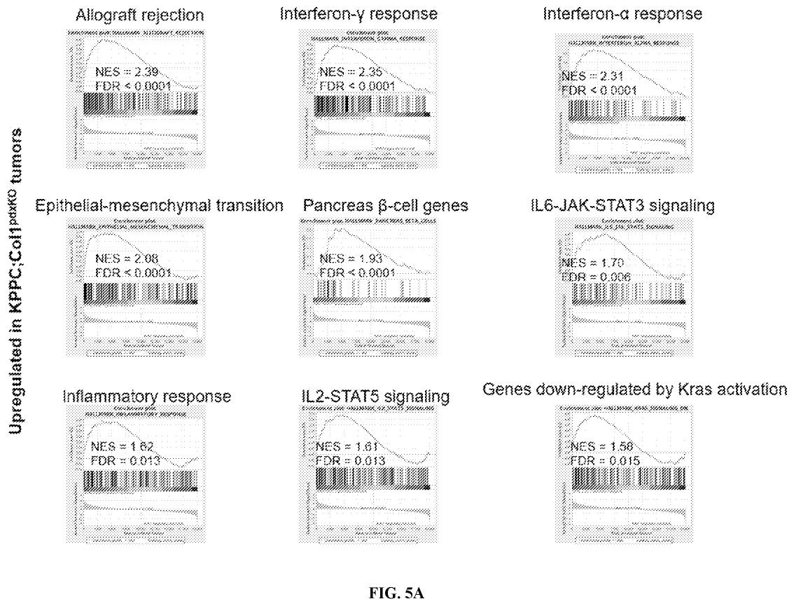

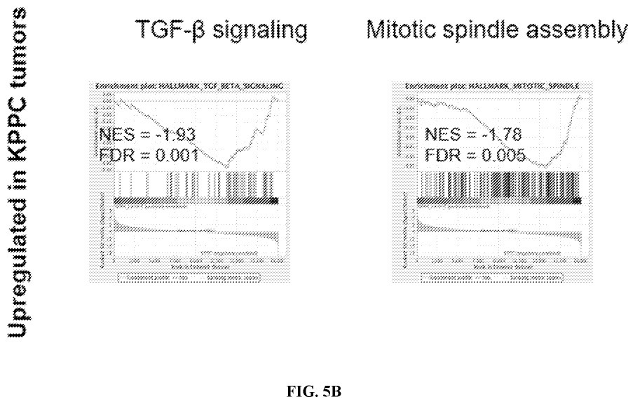

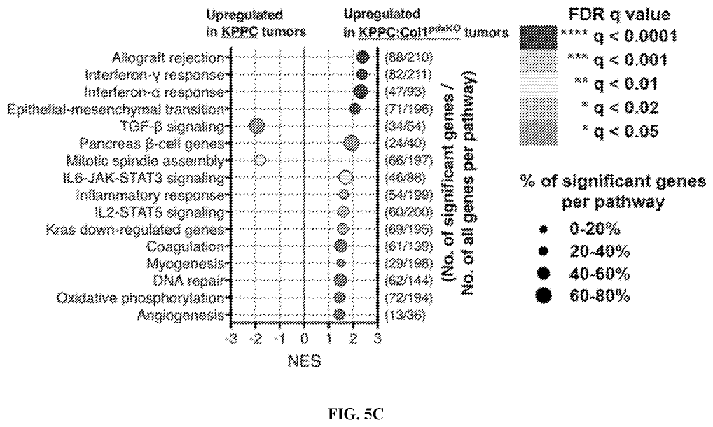

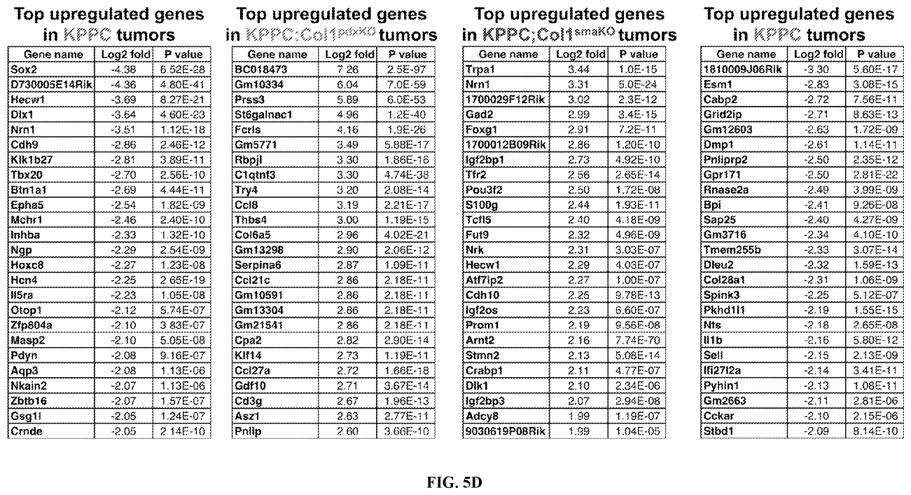

RNA-Sequencing analysis was conducted in total RNA from tumor tissues of age-matching KPPC;Col1^{pdxKO} mice (n=5) and KPPC control mice (n=4) at the same age of 53 days. Gene set enrichment analysis (GSEA) revealed significantly upregulated transcriptional signatures in hallmark pathways related to interferon response, inflammatory response, mesenchymal signature, IL6/IL2 pathways, and Kras-downregulated signaling in KPPC;Col1^{pdxKO} tumors (FIGS. 5C&D). These results demonstrate the elevated immune response, immune infiltration, and stromal response upon Col1 deletion in cancer cells, which further contributes to the suppressed PDAC progression. This is surprising given that inflammation has been shown to directly contribute to PDAC development, whereas these results reveal upregulated inflammatory pathways in delayed PDAC development with better histology upon Col1 deletion in cancer cells. In contrast, GSEA also revealed significantly upregulated transcriptional signatures in hallmark pathways related to TGF-β signaling and mitotic spindle regulation in KPPC tumors, consistent with the more advanced PDAC stage in these tumors.

RNA-Sequencing analysis was also conducted in total RNA from KPPC and KPPC;Col1^{pdxKO} primary cancer cell lines, respectively. Significant changes in gene expression profile were observed upon the deletion of Col1a1 in cancer cells (FIGS. 5H&I).

### Example 6—PDAC Cancer Cells Exhibited Significant Phenotypical Changes Upon Col1 Deletion

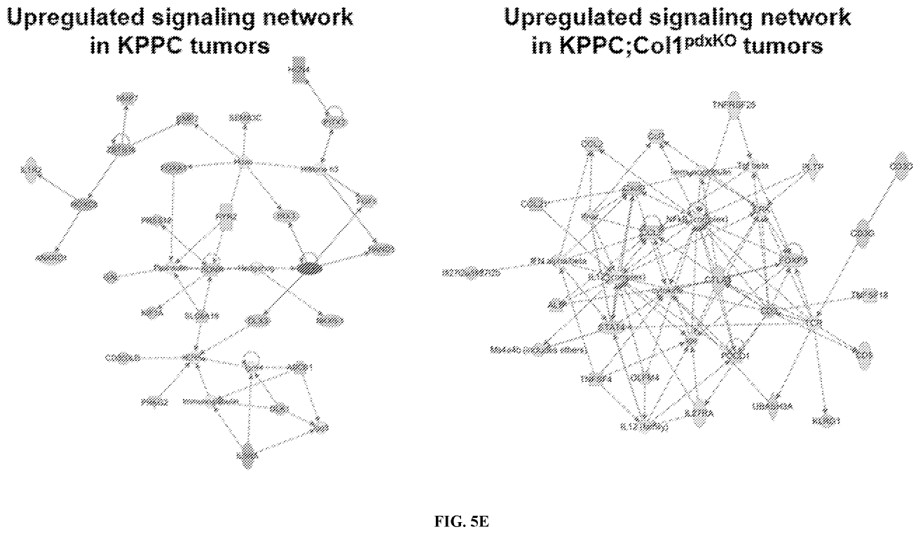

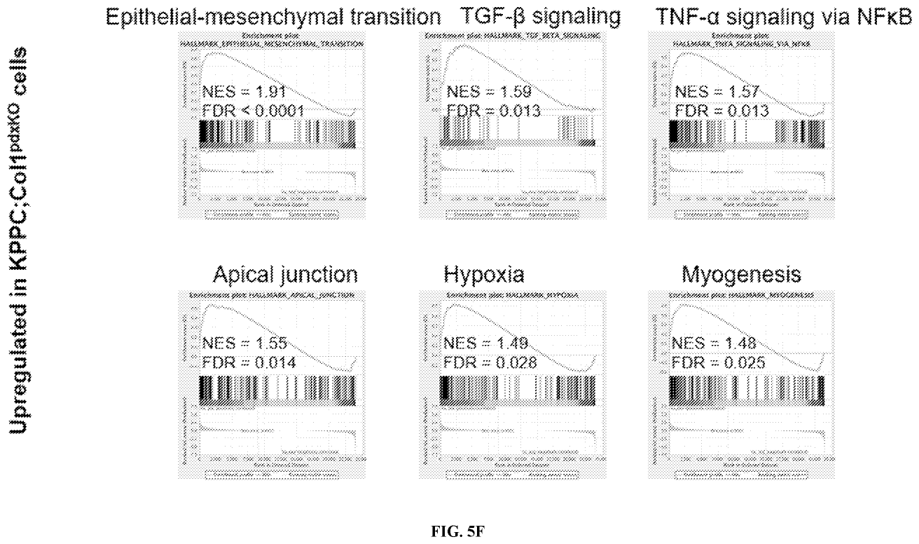

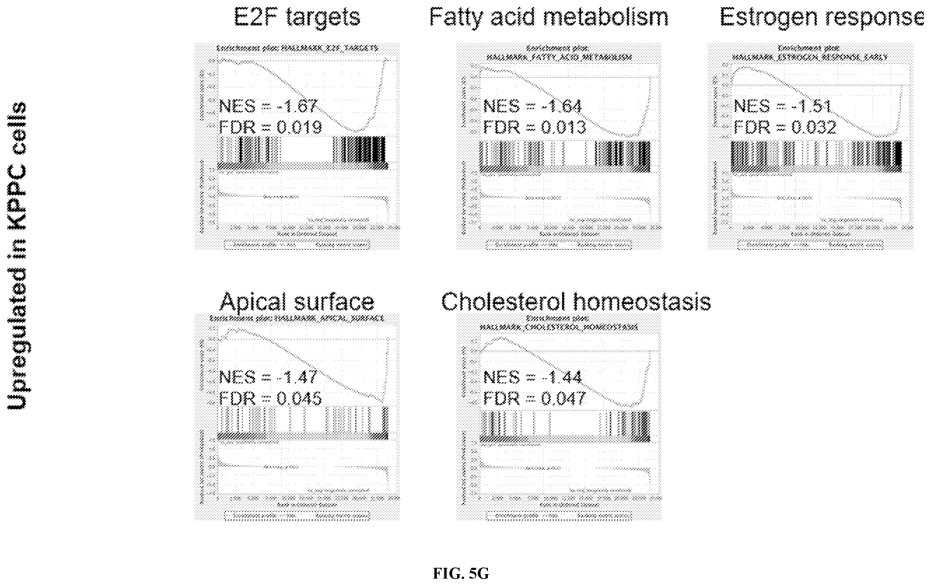

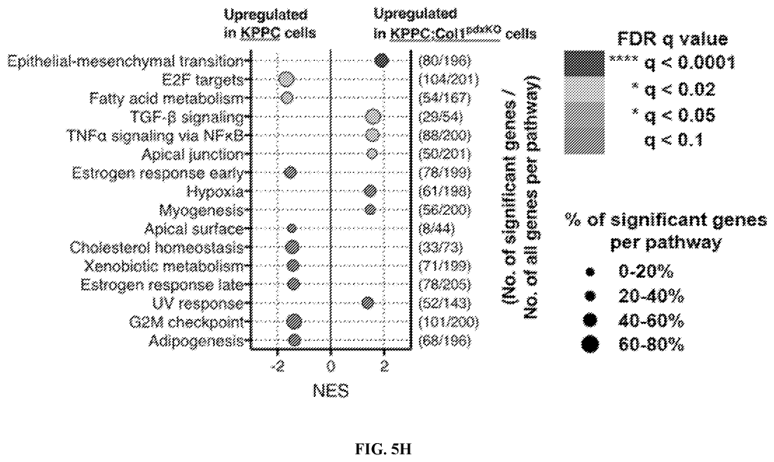

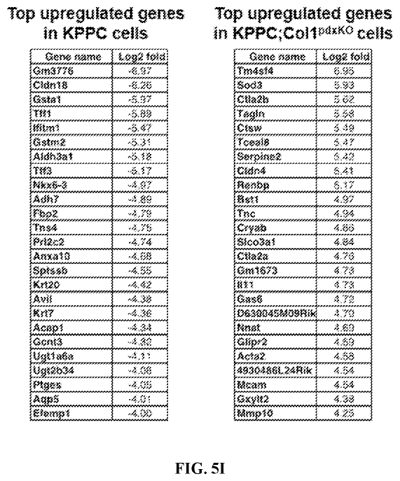

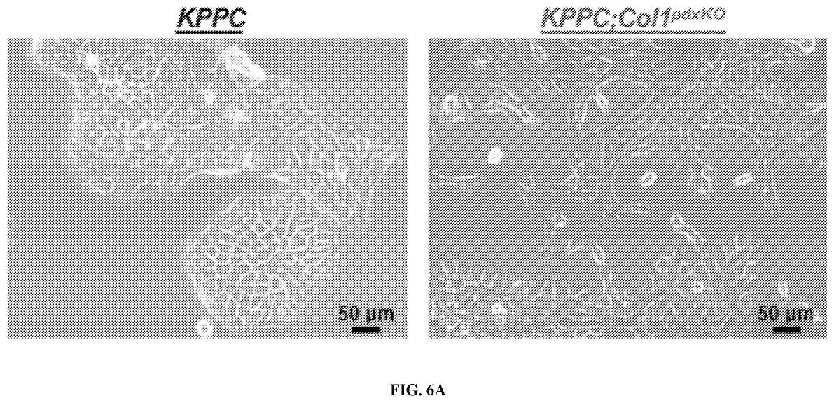

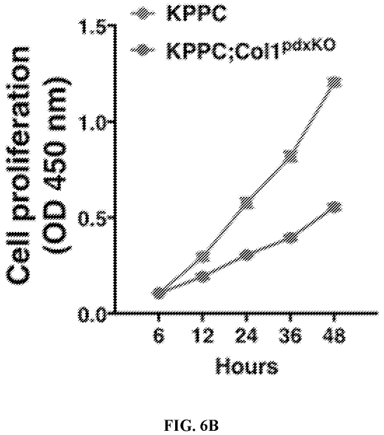

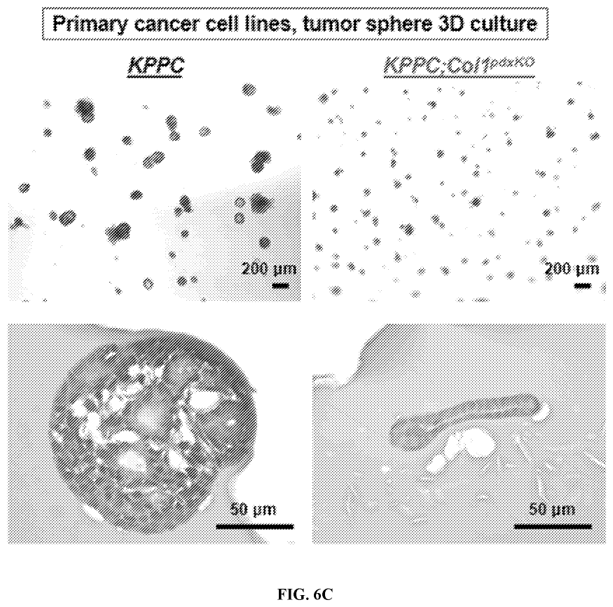

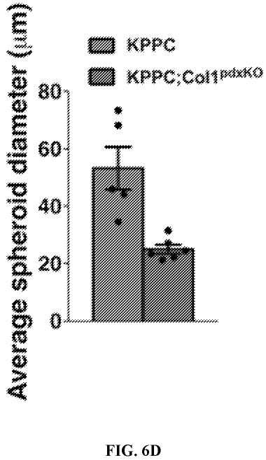

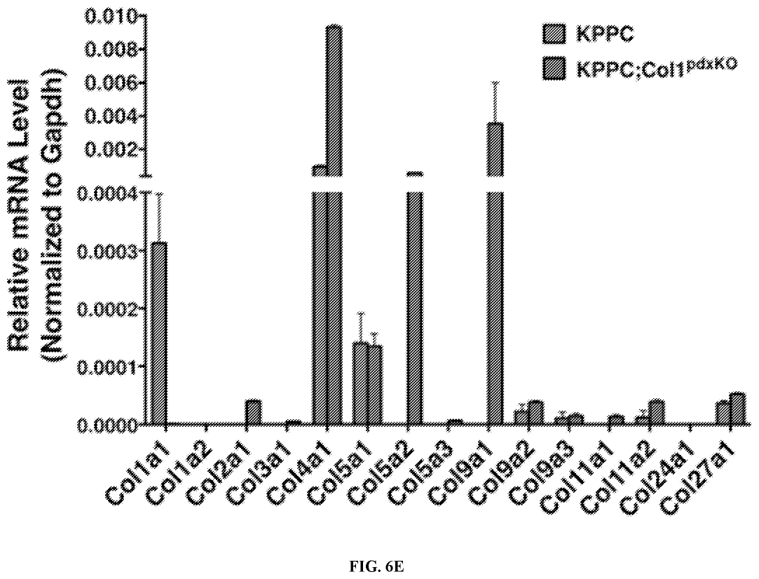

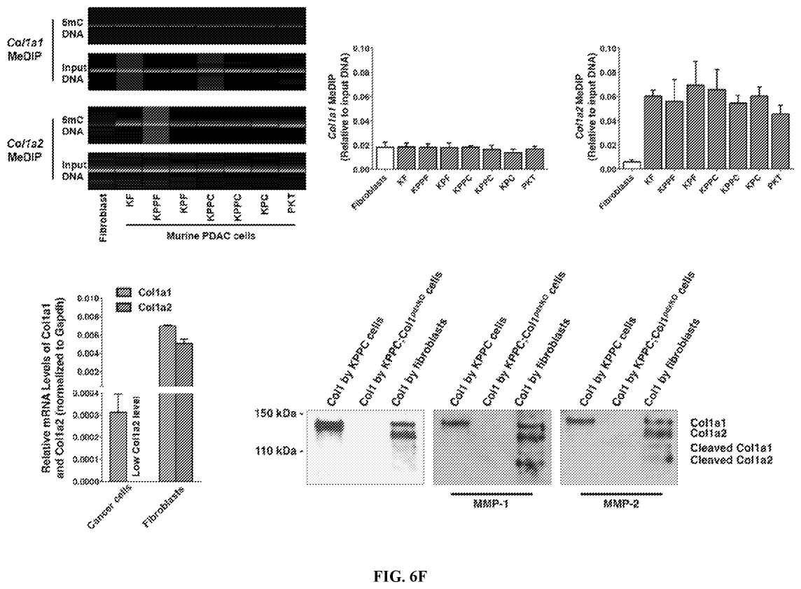

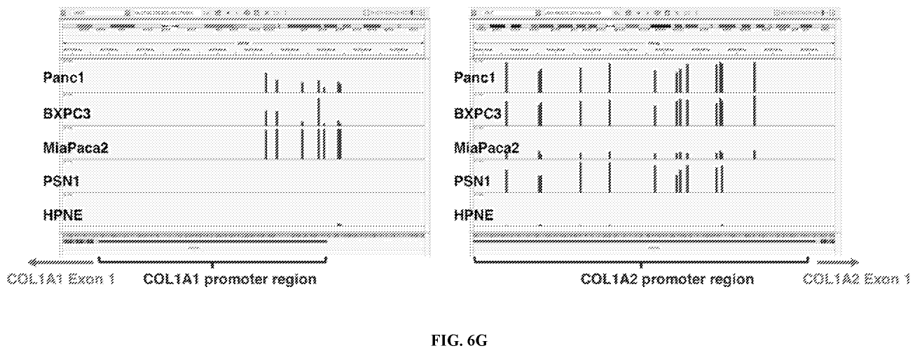

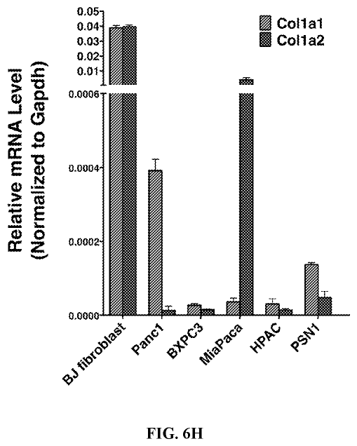

Primary pancreatic cancer cell lines were also established from tumor tissues of KPPC;Col1^{pdxKO} mouse and KPPC control mouse, respectively. As shown in FIG. 6A, KPPC;Col1^{pdxKO} primary cancer cell line revealed decreased cell adhesion and distinct cell morphology (spindle-shaped cells) when compared with KPPC cancer cell line (cobblestone-shaped cells growing in colonies).

The proliferation of KPPC;Col1^{pdxKO} primary cancer cell line in 2D cell culture system was significantly slower than that of KPPC cancer cell line (MIT; FIG. 6B). KPPC;Col1^{pdxKO} primary cancer cell line also revealed impeded ability of tumor sphere formation in 3D Matrigel (FIGS. 6C&D).

Interestingly, primary PDAC cells from KPPC mice revealed detectable expression levels of Col1a1 but not Col1a2 (FIG. 6E), consistent with the notion that cancer cells of several cancer types express Col1 homotrimer (α1)3 because of the DNA hypermethylation of Col1a2 gene and loss of Col1a2 expression. Noticeably, primary PDAC cells from KPPC;Col1^{pdxKO} mice exhibited efficient knockdown of Col1a1 but significantly elevated expression levels of Col4a1, Col5a2, and Col9a1, presumably due to a compensating mechanism (FIG. 6E).

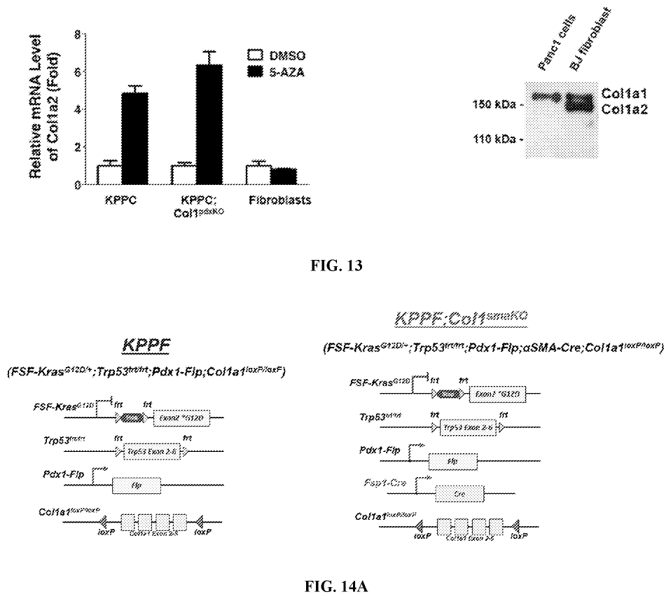

To examine the DNA methylation level of Col1a2 gene, methylated DNA immunoprecipitation (MeDIP) assay was conducted in multiple PDAC cell lines established from tumors of various PDAC transgenic mouse models including KF, KPF, KPPF, KPC, and KPPC strains (FIG. 6F). MeDIP assay revealed the DNA hypermethylation of Col1a2 gene but not Col1a1 gene in these murine primary PDAC cells (FIG. 6F) as well as consistent observations in human cancer cell lines (FIG. 12C). In contrast, fibroblasts isolated from KPPC mouse tumors revealed very low level of Col1a2 DNA methylation (FIG. 6F) and expressed high levels of both Col1a1 and Col1a2 at the similar level (FIG. 13). In addition, the treatment of de-methylation agent 5-Azacitidine partially recovered the expression level of Col1a2 in cancer cells, but not in fibroblasts (FIG. 13). These results confirmed the suppressed expression of Col1a2 in cancer cells by DNA hypermethylation.

Next, KPPC and KPPC;Col1^{pdxKO} cancer cell lines were examined for cell proliferation upon the treatment with various concentrations of Col1. Interestingly, Col1 treatment marginally inhibited the cell proliferation of KPPC cancer cell line, but significantly inhibited the proliferation of KPPC;Col1^{pdxKO} cancer cell line (FIG. 12D). This is intriguing given that the Col1 isolated from rat tail tendon is heterotrimeric in contrast to the cancer cell-derived homotrimeric Col1. These observations are consistent with the results that myofibroblast-derived heterotrimeric Col1 suppresses the growth of pancreatic tumor, especially when the cancer cells are deleted for their own Col1a1 homotrimer. These results indicate the distinct functions of cancer cell-derived Col1 homotrimers and normal tissue-derived Col1 heterotrimers.

Interestingly, KPPC;Col1^{pdxKO} cancer cells revealed an unexpected increase of DDR1, one of the receptors for Col1 in epithelial cells and cancer cells. Next, the effect of DDR1 inhibitor (3-(2-(pyrazolo(1,5-a)pyrimidin-6-yl)-ethynyl)benzamide compound (7rh) was tested on both KPPC and KPPC;Col1^{pdxKO} cancer cell lines in the presence of Col1 supplement (Col1 heterotrimer solution from rat tail). Interestingly, KPPC;Col1^{pdxKO} cancer cells responded to 7rh differently from KPPC control cells, showing a prominent cell growth increase at the lower dosages of 7rh. This result indicates that 7rh at low concentrations can reverse the growth inhibition on KPPC;Col1^{pdxKO} cancer cells by supplied Col1 heterotrimer solution, while 7rh at higher concentrations can eventually block this signaling pathway and significantly inhibit cell proliferation.

### Example 7—Col1 Deletion in Fsp1-Expressing Fibroblast Subpopulation Did not Influence PDAC Progression

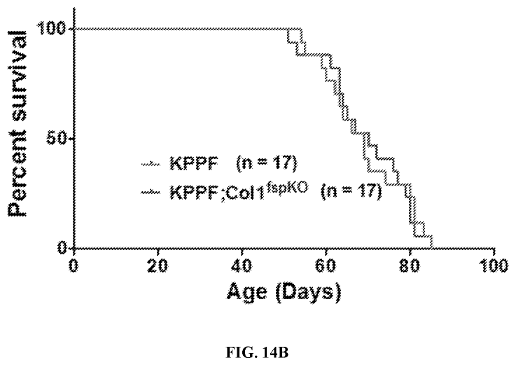

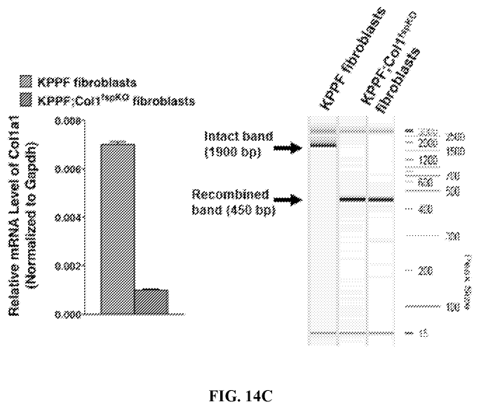

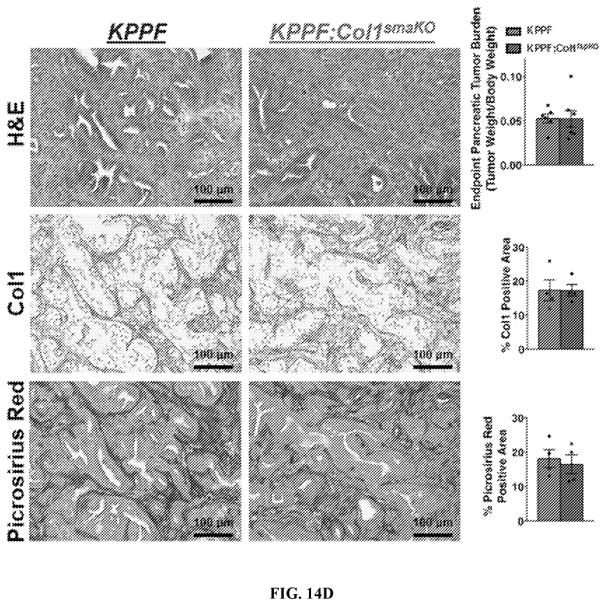

Given the previous observations showing that Col1 deletion in αSMA-expressing activated PSCs results in accelerated PanIN development, it was next asked whether Col1 deletion in another fibroblast subpopulation within PDAC could also lead to a similar phenotype. The KPPF;Col1^{fspKO} (FSF-Kras^{G12D/+};Trp53^{frt/frt};Pdx1-Flp;Fsp1-Cre;Col1a1^{loxP/loxP}) mice (FIG. 14A) were generated using the fibroblast-specific Fsp1-Cre transgene. Interestingly, KPPF;Col1^{fspKO} mice, allowing for Col1 deletion in Fsp1-expressing fibroblasts revealed no difference in animal survival and PDAC progression when compared with KPPC littermate control mice (FIG. 14B). The KPPF;Col1^{fspKO} system efficiently deleted Col1 in Fsp1-expressing fibroblasts, as confirmed in isolated primary Fsp1-expressing fibroblasts (FIG. 14C). However, the overall level of Col1 in PDAC tissue was not significantly decreased in KPPF;Col1^{fspKO} mice when compared with KPPF control mice (FIG. 14D), indicating that Fsp1-expressing fibroblast subpopulation may not be the major contributor of Col1 in PDAC stromal. These results support the heterogeneity of fibroblast subpopulations in PDAC microenvironment and their various contributions in collagen deposition.

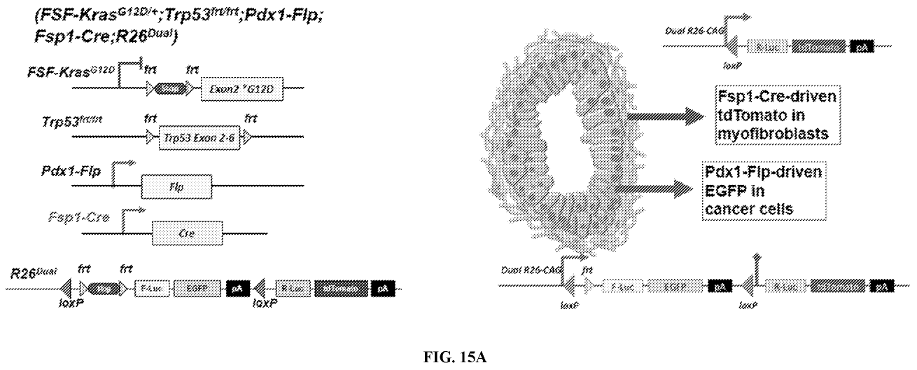

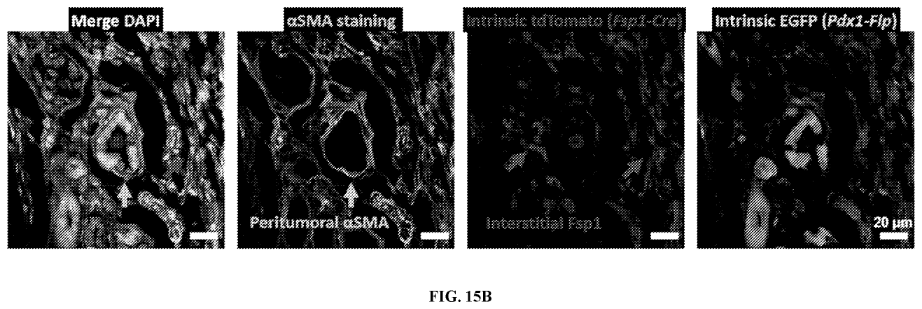

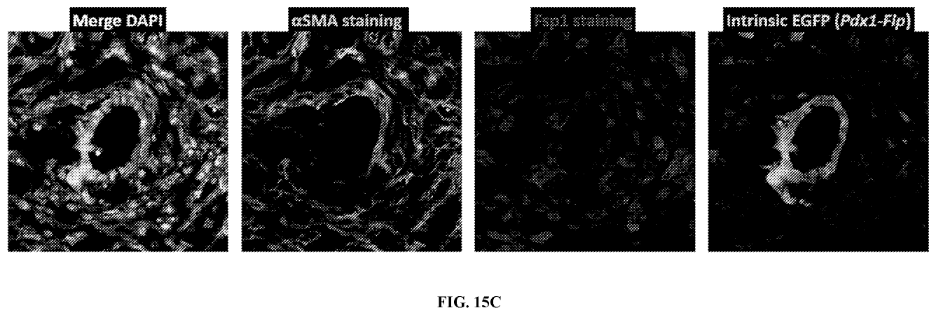

To further probe the heterogeneity of fibroblast subpopulations, KPPF;Fsp1-Cre;R26^{Dual} mice (FIG. 15A), in which Pdx1-lineage cancer cells express EGFP and Fsp1-lineage fibroblasts express tdTomato, were generated. The specificity and efficacy of the Fsp1-Cre transgene in this mouse model was confirmed by the colocalization between Fsp1 antibody staining and Fsp1-Cre-induced tdTomato signal (FIG. 15B). Intriguingly, Fsp1-expressing fibroblasts revealed interstitial localization pattern of PDAC stroma, which was significantly distinct from the peri-tumoral localization of αSMA-expressing activated PSCs (FIG. 15B). Such minimal colocalization between Fsp1 and αSMA fibroblast subpopulations was also confirmed by immunofluorescence staining using CSMA antibody and Fsp1 antibody (FIG. 15C).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Apte M V, Pirola R C, Wilson J S. 2012. Pancreatic stellate cells: a starring role in normal and diseased pancreas. Front Physiol 3: 344.
Armstrong T, Packham G, Murphy L B, Bateman A C, Conti J A, Fine D R, Johnson C D, Benyon R C, Iredale J P. 2004. Type I collagen promotes the malignant phenotype of pancreatic ductal adenocarcinoma. Clinical cancer research: an official journal of the American Association for Cancer Research 10: 7427-7437.
Bachem M G, Schunemann M, Ramadani M, Siech M, Beger H, Buck A, Zhou S, Schmid-Kotsas A, Adler G. 2005. Pancreatic carcinoma cells induce fibrosis by stimulating proliferation and matrix synthesis of stellate cells. Gastroenterology 128: 907-921.
Bhowmick N A, Chytil A, Plieth D, Gorska A E, Dumont N, Shappell S, Washington M K, Neilson E G, Moses H L. 2004. TGF-beta signaling in fibroblasts modulates the oncogenic potential of adjacent epithelia. Science 303: 848-851.
Chen Z, Trotman L C, Shaffer D, Lin H K, Dotan Z A, Niki M, Koutcher J A, Scher H I, Ludwig T, Gerald W et al. 2005. Crucial role of p53-dependent cellular senescence in suppression of Pten-deficient tumorigenesis. Nature 436: 725-730.
Egeblad M, Rasch M G, Weaver V M. 2010. Dynamic interplay between the collagen scaffold and tumor evolution. Curr Opin Cell Biol 22: 697-706.
Fujita H, Ohuchida K, Mizumoto K, Egami T, Miyoshi K, Moriyama T, Cui L, Yu J, Zhao M, Manabe T et al. 2009. Tumor-stromal interactions with direct cell contacts enhance proliferation of human pancreatic carcinoma cells. Cancer science 100: 2309-2317.
Haber P S, Keogh G W, Apte M V, Moran C S, Stewart N L, Crawford D H, Pirola R C, McCaughan G W, Ramm G A, Wilson J S. 1999. Activation of pancreatic stellate cells in human and experimental pancreatic fibrosis. The American journal of pathology 155: 1087-1095.
Han S, Makareeva E, Kuznetsova N V, DeRidder A M, Sutter M B, Losert W, Phillips C L, Visse R, Nagase H, Leikin S. 2010. Molecular mechanism of type I collagen homotrimer resistance to mammalian collagenases. The Journal of biological chemistry 285: 22276-22281.
Han S, McBride D J, Losert W, Leikin S. 2008. Segregation of type I collagen homo- and heterotrimers in fibrils. J Mol Biol 383: 122-132.
Hingorani S R, Wang L, Multani A S, Combs C, Deramaudt T B, Hruban R H, Rustgi A K, Chang S, Tuveson D A. 2005. Trp53R172H and KrasG12D cooperate to promote chromosomal instability and widely metastatic pancreatic ductal adenocarcinoma in mice. Cancer cell 7: 469-483.
Kalluri R. 2016. The biology and function of fibroblasts in cancer. Nature reviews Cancer 16: 582-598.
Kopp J L, von Figura G, Mayes E, Liu F F, Dubois C L, Morris J Pt, Pan F C, Akiyama H, Wright C V, Jensen K et al. 2012. Identification of Sox9-dependent acinar-to-ductal reprogramming as the principal mechanism for initiation of pancreatic ductal adenocarcinoma. Cancer cell 22: 737-750.
Laklai H, Miroshnikova Y A, Pickup M W, Collisson E A, Kim G E, Barrett A S, Hill R C, Lakins J N, Schlaepfer D D, Mouw J K et al. 2016. Genotype tunes pancreatic ductal adenocarcinoma tissue tension to induce matricellular fibrosis and tumor progression. Nature medicine 22: 497-505.
LeBleu V S, Taduri G, O'Connell J, Teng Y, Cooke V G, Woda C, Sugimoto H, Kalluri R. 2013. Origin and function of myofibroblasts in kidney fibrosis. Nature medicine 19: 1047-1053.
Lee C L, Moding E J, Huang X, Li Y, Woodlief L Z, Rodrigues R C, Ma Y, Kirsch D G. 2012. Generation of primary tumors with Flp recombinase in FRT-flanked p53 mice. Disease models & mechanisms 5: 397-402.
Lohler J, Timpl R, Jaenisch R. 1984. Embryonic lethal mutation in mouse collagen I gene causes rupture of blood vessels and is associated with erythropoietic and mesenchymal cell death. Cell 38: 597-607.
Makareeva E, Han S, Vera J C, Sackett D L, Holmbeck K, Phillips C L, Visse R, Nagase H, Leikin S. 2010. Carcinomas contain a matrix metalloproteinase-resistant isoform of type I collagen exerting selective support to invasion. Cancer research 70: 4366-4374.
Mueller M M, Fusenig N E. 2004. Friends or foes—bipolar effects of the tumour stroma in cancer. Nature reviews Cancer 4: 839-849.
Neesse A, Algul H, Tuveson D A, Gress T M. 2015. Stromal biology and therapy in pancreatic cancer: a changing paradigm. Gut 64: 1476-1484.
Ohlund D, Elyada E, Tuveson D. 2014. Fibroblast heterogeneity in the cancer wound. The Journal of experimental medicine 211: 1503-1523.
Ohlund D, Handly-Santana A, Biffi G, Elyada E, Almeida A S, Ponz-Sarvise M, Corbo V, Oni T E, Hearn S A, Lee E J et al. 2017. Distinct populations of inflammatory fibroblasts and myofibroblasts in pancreatic cancer. The Journal of experimental medicine 214: 579-596.
Olive K P, Jacobetz M A, Davidson C J, Gopinathan A, McIntyre D, Honess D, Madhu B, Goldgraben M A, Caldwell M E, Allard D et al. 2009. Inhibition of Hedgehog signaling enhances delivery of chemotherapy in a mouse model of pancreatic cancer. Science 324: 1457-1461.
Ozdemir B C, Pentcheva-Hoang T, Carstens J L, Zheng X, Wu C C, Simpson T R, Laklai H, Sugimoto H, Kahlert C, Novitskiy S V et al. 2014. Depletion of carcinoma-associated fibroblasts and fibrosis induces immunosuppression and accelerates pancreas cancer with reduced survival. Cancer cell 25: 719-734.
Provenzano P P, Cuevas C, Chang A E, Goel V K, Von Hoff D D, Hingorani S R. 2012. Enzymatic targeting of the stroma ablates physical barriers to treatment of pancreatic ductal adenocarcinoma. Cancer cell 21: 418-429.
Rhim A D, Oberstein P E, Thomas D H, Mirek E T, Palermo C F, Sastra S A, Dekleva E N, Saunders T, Becerra C P, Tattersall I W et al. 2014. Stromal elements act to restrain, rather than support, pancreatic ductal adenocarcinoma. Cancer cell 25: 735-747.
Schonhuber N, Seidler B, Schuck K, Veltkamp C, Schachtler C, Zukowska M, Eser S, Feyerabend T B, Paul M C, Eser P et al. 2014. A next-generation dual-recombinase system for time- and host-specific targeting of pancreatic cancer. Nature medicine 20: 1340-1347.
Sengupta P K, Smith E M, Kim K, Murnane M J, Smith B D. 2003. DNA hypermethylation near the transcription start site of collagen alpha2(I) gene occurs in both cancer cell lines and primary colorectal cancers. Cancer research 63: 1789-1797.
Seymour P A, Freude K K, Tran M N, Mayes E E, Jensen J, Kist R, Scherer G, Sander M. 2007. SOX9 is required for maintenance of the pancreatic progenitor cell pool. Proceedings of the National Academy of Sciences of the United States of America 104: 1865-1870.
Xue C, Plieth D, Venkov C, Xu C, Neilson E G. 2003. The gatekeeper effect of epithelial-mesenchymal transition regulates the frequency of breast cancer metastasis. Cancer research 63: 3386-3394.

1. An antibody or antibody fragment that binds to α1 homotrimeric type I collagen.

2. (canceled)

3. The antibody or antibody fragment of claim 1, wherein the antibody or antibody fragment has an affinity for α1 homotrimeric type I collagen that is at least five times higher than an affinity for α1/α2/α1 heterotrimeric type I collagen.

4. (canceled)

5. (canceled)

6. antibody or antibody fragment of claim 1, wherein the antibody or antibody fragment is a bispecific antibody that binds to both α1 homotrimeric type I collagen and CD3.

7. (canceled)

8. (canceled)

9. The antibody or antibody fragment of claim 1, wherein the antibody or antibody fragment is conjugated to a cytotoxic agent or a diagnostic agent.

10. (canceled)

11. A hybridoma or engineered cell encoding the antibody or antibody fragment of claim 1.

12. A pharmaceutical formulation comprising the antibody or antibody fragment of claim 1.

13. A method of treating a patient in need thereof, the method comprising administering an effective amount of the pharmaceutical formulation of claim 12.

14. The method of claim 13, wherein the patient has a cancer, a fibroid disease, keloids, organ fibrosis, Crohn's disease, strictures, colitis, psoriasis, or a connective tissue disorder.

15. (canceled)

16. (canceled)

17. The method of claim 14, wherein the patient has a cancer.

18. (canceled)

19. The method of claim 17, wherein said cancer patient has been determined to express an elevated level of α1 homotrimeric type I collagen.

20. The method of claim 17, wherein the cancer is a pancreatic cancer.

21. (canceled)

22. (canceled)

23. The method of claim 17, further comprising administering at least a second anti-cancer therapy, wherein the second anti-cancer therapy is a chemotherapy, immunotherapy, radiotherapy, gene therapy, surgery, hormonal therapy, anti-angiogenic therapy or cytokine therapy.

24. (canceled)

25. A chimeric antigen receptor (CAR) polypeptide comprising, from N- to C-terminus, an antigen binding domain; a hinge domain; a transmembrane domain and an intracellular signaling domain, wherein the CAR polypeptide binds to an α1 homotrimeric type I collagen.

26.-28. (canceled)

29. The polypeptide of claim 25, wherein the antigen binding domain has an affinity for α1 homotrimeric type I collagen that is at least five times higher than an affinity for α1/α2/α1 heterotrimeric type I collagen.

30.-33. (canceled)

34. A nucleic acid molecule encoding the CAR polypeptide of claim 25.

35. (canceled)

36. An isolated immune effector cell comprising the nucleic acid molecule of claim 34.

37. (canceled)

38. The cell of claim 36, wherein the cell is a T cell or an NK cell.

39. (canceled)

40. (canceled)

41. A pharmaceutical composition comprising the cell of claim 36 in a pharmaceutically acceptable carrier.

42. A method of treating a subject comprising administering an anti-tumor effective amount of the pharmaceutical composition of claim 41.

43.-45. (canceled)

46. The method of claim 42, wherein the subject has cancer.

47. The method of claim 46, wherein the cancer is pancreatic cancer.

48. The method of any one of claims 42-47, further comprising administering a demethylating drug prior to administering the pharmaceutical composition.

49. The method of claim 48, wherein the demethylating drug reverses Col1A2 hypermethylation.

50.-59. (canceled)

60. A method of identifying diseased tissue, the method comprising contacting tissue obtained from the subject with the antibody or antibody fragment of any claim 1 and detecting the binding of the antibody or antibody fragment to the tissue.

61. The method of claim 60, wherein the diseased tissue is from a subject having cancer, a fibroid disease, keloids, organ fibrosis, Crohn's disease, strictures, colitis, psoriasis, or a connective tissue disorder.

62. (canceled)

63. (canceled)

64. A method of classifying a patient having pancreatic ductal adenocarcinoma, the method comprising determining a type I collagen/CK19 ratio in a cancer tissue obtained from the subject, wherein a ratio that is lower than a ratio in a reference normal tissue indicates that the patient has a more advanced disease status.

65.-78. (canceled)
